(19) United States
(12) Patent Application Publication
(10) Pub. No.: US 2006/0123489 A1
(43) Pub. Date: Jun. 8, 2006

(54) Sex-specific automated sorting of non-human animals

(75) Inventors: Joost Schulte, Newton, MA (US); Hsin-Pei Shih, Watertown, MA (US); Chris Cummings, Brookline, MA (US)

Correspondence Address: PALMER & DODGE, LLP;KATHLEEN M. WILLIAMS, 111 HUNTINGTON AVENUE, BOSTON, MA, 02199, US

(73) Assignee: EnVivo Pharmaceuticals, Inc.

(21) Appl. No.: 11/004,732

(22) Filed: Dec. 3, 2004

(51) Int. Cl. A01K 67/033; A01K 67/027

(52) U.S. Cl. 800/8; 800/20

(57) ABSTRACT

The present invention relates to populations of male and female non-human animals which can be induced to produce single sex populations, and which may comprise a heterologous gene of interest, preferably a neurodegenerative disease gene, which is expressed in a tissue specific manner. The invention also relates to methods for sorting a mixed population of non-human animal embryos and/or larvae into a single sex population.

### BACKGROUND

Neurodegenerative diseases are among some of the most devastating diseases afflicting humans. Examples of neurodegenerative diseases include Alzheimer's Disease, Parkinson's Disease, Huntington's Disease and spinocerebellar ataxia (SCA). However, the discovery and development of therapeutics for disorders of the central nervous system (CNS), particularly for neurodegenerative diseases, has historically been very difficult.

Due to the ease and speed with which genetic studies can be pursued in Drosophila, these models have been especially useful in identifying genes that modify disease. The investigation of pathogenic mechanisms in neurodegenerative disease has been facilitated by the recent development of disease models in Drosophila. By introducing human disease genes with dominant gain-of-function mutations into Drosophila, models for a number of neurodegenerative diseases have been generated, including models for Huntington's disease and spinocerebellar ataxia (see, for example, Chan et al. (2000) Cell Death Differ. 7:1075-1080; Feany et al. (2000) Nature 404:394-398; Femandez-Funez et al. (2000) Nature 408:101-106; Fortini et al. (2000) Trends Genet. 16:161-167; Jackson et al. (1998) Neuron 21:633-642; Kazemi-Esfarjani et al. (2000) Science 287:1837-1840; Warrick et al. (1998) Cell 93:939-949. Transgenic technology has advanced in Drosophila such that cell or tissue specific expression can be achieved by placing the human gene under control of the GAL4/UAS transcriptional activation system from yeast (Brand et al. (1993) Development 118:401-415).

In some cases, expression of the transgene recapitulates one or more neuropathological features of the human disease. For example, in a Drosophila model for Parkinson's disease produced by neuronal expression of human mutated alpha-synuclein, age-dependent, progressive degeneration of dopamine-containing cells is seen accompanied by the presence of Lewy bodies that resemble those seen in the human disease, both by their immunoreactivity for alpha-synuclein and by their appearance in the electron microscope (Feany et al. (2000)). In the SCA1^{82Q} flies, expression of the mutated human ataxin-1 (associated with SCA) is accompanied by adult-onset degeneration of neurons, with nuclear inclusions that are immunologically positive for the mutated protein, as well as ubiquitin, Hsp70 and proteosome components (Femandez-Funez et al. (2000)). In the case of Huntington's disease, expression of exon-1 of huntingtin, containing an expanded polyglutamine repeat, causes a progressive degeneration, whose time of onset and severity are linked to the length of the repeat, as is seen in the human disease (Marsh et al. (2000) Hum. Mol. Genet. 9:13-25).

Although great advances have been made in understanding the biological basis of neurological disorders, this scientific progress has generally not yet been translated into effective new treatments for these devastating disorders. There remains a tremendous need for new methods of drug discovery for CNS disorders, particularly for neurodegenerative diseases.

There is also a need in the art for a high-throughput method for the generation of single sex populations of Drosophila for the study of neurodegenerative disease. For example, many studies of neurodegenerative pathology in Drosophila utilize exclusively male or female flies in assays such as climbing assays (i.e., a negative geotaxis assay). The traditional method for generating a single sex population of organisms for such studies is to manually sort male and female organisms (e.g., flies), which is labor intensive and time consuming, and is not easily adapted to high throughput assays.

Methods for inducing heat shock responsive, head involution defective mediated, sex-selective death of Drosophila have been taught in the art (Grether et al., 1995, Genes and Development 9:1694; Moore et al., 1998, Development 125:667; U.S. Pat. No. 6,235,524; and WO94/16071). In contrast to the teachings in the art, however, the present invention has provided a method which integrates the use of a pro-apoptotic gene (e.g., head involution defective) with other genetic elements to provide a sortable population of non-human animals which express a heterologous gene of interest. One embodiment of the invention utilizes COPAS flow cytometry sorting to isolate a pure single sex population. The COPAS technology described herein is known in the art and has been described for the fluorescent sorting of Drosophila based on GFP expression (Union Biometrica; 45^{th} Annual Drosophila Research Conference (2004). Union Biometrica, however, describes the expression of GFP linked to the X chromosome under the control of a sex specific promoter (Sxl P_{E}). The present invention has an advantage over the sorting taught by Union Biometrica, in that the present invention does not require the use of a sex-specific promoter system, which can be time consuming, and requires additional genetic manipulation. The present invention instead uses the combination of sex-specific, apoptosis-induced culling of a population of non-human animals and genetic sorting of genetically modified fluorescent marker elements to produce single sex populations of non-human animals.

### SUMMARY OF THE INVENTION

The present invention relates, in part, to the use of inducible pro-apoptotic genes for the generation of single sex populations of Drosophila, particularly Drosophila embryos, which may be used in subsequent assays for the identification of compounds useful in the treatment of neurodegenerative disorders. The present invention also provides a method for the production of single sex populations of Drosophila using flow cytometry, and thus provides a high-throughput method for the segregation of flies based on sex. It is also contemplated that the methods described herein for sex-specific sorting are not limited to Drosophila, but may be utilized for the sorting of other non-human animals of interest, provided that the non-human animal has an embryo or larval size of greater than 50 μm in diameter and preferably having at least one dimension ranging between 70 and 500 μm or larger.

The present invention encompasses a population of male and female non-human animals wherein the male animals comprises a pro-apoptotic gene operably linked to a regulatable promoter integrated into the Y chromosome, and wherein the regulatable promoter is not a heat-shock promoter.

In one embodiment, the pro-apoptotic gene is selected from the group consisting of head involution defective, reaper, grim, hid-ala, ICE, and ced-3.

In another embodiment, the animals are selected from the group consisting of Drosophila, silkworm, C. elegans, zebrafish, zooplankton, medakafly, mosquito, and xenopus.

In another embodiment, the animals are Drosophila.

The invention further encompasses a population male and female non-human animals wherein the male animals comprise a pro-apoptotic gene operably linked to a regulatable promoter integrated into the Y chromosome and further comprises, integrated into the genome of the male and female non-human animals a sequence encoding Gal4 operably linked to a neuronal or glial-specific promoter.

In one embodiment, the pro-apoptotic gene is selected from the group consisting of head involution defective, reaper, grim, hid-ala, ICE, and ced-3.

In another embodiment, the regulatable promoter is selected from the group consisting of heat shock promoter, Gal40, Gal80, Tet, and RU486.

In another embodiment, the animals are selected from the group consisting of Drosophila, silkworm, C. elegans, zebrafish, zooplankton, medakafly, mosquito, and xenopus.

In another embodiment, the animals are Drosophila.

The invention also encompasses a population of insects comprising male and female insects wherein the male insects comprises a pro-apoptotic gene operably linked to a regulatable promoter integrated into the Y chromosome and further comprises, integrated into the genome of the male and female insects a sequence encoding Gal4 operably linked to a neuronal or glial-specific promoter.

In one embodiment, the pro-apoptotic gene is selected from the group consisting of head involution defective, reaper, grim, hid-ala, ICE, and ced-3.

In another embodiment, the regulatable promoter is selected from the group consisting of heat shock promoter, Gal40, Gal80, Tet, and RU486.

In another embodiment, the insects are selected from the group consisting of Drosophila, silkworm, and mosquito.

In another embodiment, the insects are Drosophila.

The invention also encompasses a population of Drosophila comprising male and female Drosophila wherein the male Drosophila comprise a pro-apoptotic gene operably linked to a regulatable promoter integrated into the Y chromosome and further comprises, integrated into the genome of the male and female Drosophila a sequence encoding Gal4 operably linked to a neuronal or glial-specific promoter.

In one embodiment, the pro-apoptotic gene is selected from the group consisting of head involution defective, reaper, grim, hid-ala, ICE, and ced-3.

In another embodiment, the regulatable promoter is selected from the group consisting of heat shock promoter, Gal40, Gal80, Tet, and RU486.

The invention also encompasses a population of male and female non-human animals wherein the female non-human animals comprises an attached-X chromosome, and wherein a pro-apoptotic gene is integrated into the attached-X chromosome.

In one embodiment, the pro-apoptotic gene is selected from the group consisting of head involution defective, reaper, grim, hid-ala, ICE, and ced-3.

In another embodiment, the pro-apoptotic gene is operably linked to a regulatable promoter.

In another embodiment, the regulatable promoter is selected from the group consisting of heat shock promoter, Gal40, Gal80, Tet, and RU486.

In another embodiment, the male non-human animals of the population comprise a sequence encoding a fluorescent protein integrated into the X chromosome.

In another embodiment, the animals further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the non-human animals are selected from the group consisting of Drosophila, silkworm, C. elegans, zebrafish, zooplankton, medaka, mosquito, and xenopus.

In another embodiment, the male and female non-human animals further comprises an upstream activator sequence operably linked to a neurodegenerative disease gene.

The invention also encompasses a population of male and female non-human animals wherein the female animals comprises an attached-X chromosome, and wherein a pro-apoptotic gene is integrated into the attached-X chromosome, and wherein the male and female animals further comprise an upstream activator sequence operably linked to a heterologous gene of interest.

In one embodiment, the pro-apoptotic gene is selected from the group consisting of head involution defective, reaper, grim, hid-ala, ICE, and ced-3.

In another embodiment, the pro-apoptotic gene is operably linked to a regulatable promoter.

In another embodiment, the regulatable promoter is selected from the group consisting of heat shock promoter, Gal40, Gal80, Tet, and RU486.

In another embodiment, the male animals of the population comprise a sequence encoding a fluorescent protein integrated into the X chromosome.

In another embodiment, the animals further comprise, integrated into the X chromosome, a female sterile mutation.

In another embodiment, the female sterile mutation is selected from the group consisting of fs(1)K10, JA127, JC105, EC205, EA130, RC63, VA296DF942, DC776, HA90, L271, VA172, JC155, DC798, HF330, HF311, ED226, EF462, D62, D72, EA75, gt^{xll}, and fs(1)pcx.

In another embodiment, the animals are selected from the group consisting of Drosophila, silkworm, C. elegans, zebrafish, zooplankton, medaka, mosquito, and xenopus.

The invention also encompasses a population of male and female non-human animals wherein the female animals comprises an attached-X chromosome, and wherein a pro-apoptotic gene is integrated into the attached-X chromosome, and wherein the male and female animals further comprise an upstream activator sequence operably linked to a heterologous gene of interest, and further comprises a sequence encoding a fluorescent protein integrated into a sex chromosome.

In one embodiment, the pro-apoptotic gene is selected from the group consisting of head involution defective, reaper, grim, hid-ala, ICE, and ced-3.

In another embodiment, the pro-apoptotic gene is operably linked to a regulatable promoter.

In another embodiment, the regulatable promoter is selected from the group consisting of heat shock promoter, Gal40, Gal80, Tet, and RU486.

In another embodiment, the animals further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the animals are selected from the group consisting of Drosophila, silkworm, C. elegans, zebrafish, zooplankton, medaka, mosquito, and xenopus.

The invention also encompasses a population of male and female non-human animals wherein the female animals comprises an attached-X chromosome, and wherein a pro-apoptotic gene is integrated into the attached-X chromosome, and wherein the male and female animals further comprise an upstream activator sequence operably linked to a heterologous gene of interest, and further comprises a sequence encoding a fluorescent protein integrated into a sex chromosome, and wherein the population of non-human animals further comprises, integrated in the X chromosome, a female sterile mutation.

In one embodiment, the pro-apoptotic gene is selected from the group consisting of head involution defective, reaper, grim, hid-ala, ICE, and ced-3.

In another embodiment, the pro-apoptotic gene is operably linked to a regulatable promoter.

In another embodiment, the regulatable promoter is selected from the group consisting of heat shock promoter, Gal40, Gal80, Tet, and RU486.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the animals are selected from the group consisting of Drosophila, silkworm, C. elegans, zebrafish, zooplankton, medaka, mosquito, and xenopus.

The invention also encompasses a population of insects comprising male and female insects wherein the female insects comprises an attached-X chromosome, and wherein a pro-apoptotic gene is integrated into the attached-X chromosome.

In one embodiment, the male insects comprise a sequence encoding a fluorescent protein integrated into the X chromosome.

In another embodiment, the pro-apoptotic gene is selected from the group consisting of head involution defective, reaper, grim, hid-ala, ICE, and ced-3.

In another embodiment, the pro-apoptotic gene is operably linked to a regulatable promoter.

In another embodiment, the regulatable promoter is selected from the group consisting of heat shock promoter, Gal40, Gal80, Tet, and RU486.

In another embodiment, the insects further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the insects are selected from the group consisting of Drosophila, silkworm, and mosquito.

In another embodiment, the male and female insects further comprises an upstream activator sequence operably linked to a neurodegenerative disease gene.

The invention also encompasses a population of Drosophila comprising male and female Drosophila wherein the female Drosophila comprises an attached-X chromosome, and wherein a pro-apoptotic gene is integrated into the attached-X chromosome.

In one embodiment, the pro-apoptotic gene is selected from the group consisting of head involution defective, reaper, grim, hid-ala, ICE, and ced-3.

In another embodiment, the pro-apoptotic gene is operably linked to a regulatable promoter.

In another embodiment, the regulatable promoter is selected from the group consisting of heat shock promoter, Gal40, Gal80, Tet, and RU486.

In another embodiment, the Drosophila further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the male Drosophila comprise a sequence encoding a fluorescent protein integrated into the X chromosome In another embodiment, the male and female insects further comprises an upstream activator sequence operably linked to a neurodegenerative disease gene.

The invention also encompasses a method for producing a population of female insects, comprising: preparing a first population of insects comprising male and female insects wherein the male insects comprise a pro-apoptotic gene operably linked to a regulatable promoter integrated into the Y chromosome; preparing a second population of insects comprising male and female insects wherein the female insects comprises an attached-X chromosome, and wherein a pro-apoptotic gene operably linked to a regulatable promoter is integrated into the attached-X chromosome, and wherein the male insects of the second population comprise a sequence encoding a fluorescent protein integrated into the X chromosome; inducing the regulatable promoter in the first and second populations of insects such that a third population of insects comprising the female insects of the first population is produced, and a fourth population of insects comprising the male insects of the second population is produced; crossing the third and fourth population of insects to produce a fifth population of insects comprising male and female insects; and selecting female insects from the fifth population of insects.

In one embodiment, the regulatable promoter is selected from the group consisting of heat shock promoter, Gal40, Gal80, Tet, and RU486.

The invention also encompasses a method for producing a population of female insects comprising a heterologous gene of interest, comprising: preparing a first population of insects comprising male and female insects wherein the male insects comprise a pro-apoptotic gene operably linked to a regulatable promoter integrated into the Y chromosome; preparing a second population of insects comprising male and female insects wherein the female insects comprises an attached-X chromosome, and wherein a pro-apoptotic gene operably linked to a regulatable promoter is integrated into the attached-X chromosome, and wherein the male insects of the second population comprise a sequence encoding a fluorescent protein integrated into the X chromosome; inducing the regulatable promoter in the first and second populations of insects such that a third population of insects comprising the female insects of the first population is produced, and a fourth population of insects comprising the male insects of the second population is produced; crossing the third and fourth population of insects to produce a fifth population of insects comprising male and female insects; and selecting female insects comprising the heterologous gene of interest form the fifth population of insects.

In one embodiment, the regulatable promoter is selected from the group consisting of heat shock promoter, Gal40, Gal80, Tet, and RU486.

In another embodiment, the male and female insects of the first population further comprises a sequence encoding yeast Gal4.

In another embodiment, the male and female insects of the second population further comprises an upstream activator sequence operably linked to the heterologous gene of interest

In another embodiment, the insects of the fifth population are insect embryos.

In another embodiment, the female insects of the fifth population express the fluorescent protein.

In another embodiment, the step selecting female insects comprising the heterologous gene of interest from the fifth population of insects comprises selecting female insects which express the fluorescent protein.

In another embodiment, the step selecting female insects comprising the heterologous gene of interest from the fifth population of insects comprises selecting female insects using flow cytometry.

In another embodiment, the flow cytometry is performed using a complex object parametric analyzer and sorter In another embodiment, the insects are selected from the group consisting of Drosophila, silkworm, and mosquito.

In another embodiment, the pro-apoptotic gene is selected from the group consisting of head involution defective, reaper, grim, hid-ala, ICE, and ced-3.

In another embodiment, the sequence encoding a fluorescent protein encodes a green fluorescent protein.

In another embodiment, the heterologous gene of interest is a neurodegenerative disease gene.

In another embodiment, the insects of the second population further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the fifth population of insects is placed in contact with rearing media comprising one or more test compounds.

The invention also encompasses a method for producing a population of female Drosophila comprising a heterologous gene of interest, comprising: preparing a first population of Drosophila comprising male and female Drosophila wherein the male Drosophila comprise a pro-apoptotic gene operably linked to a regulatable promoter integrated into the Y chromosome; preparing a second population of Drosophila comprising male and female Drosophila wherein the female Drosophila comprises an attached-X chromosome, and wherein a pro-apoptotic gene operably linked to a regulatable promoter is integrated into the attached-X chromosome, and wherein the male Drosophila of the second population comprise a sequence encoding a fluorescent protein integrated into the X chromosome; inducing the regulatable promoter in the first and second populations of Drosophila such that a third population of Drosophila comprising the female Drosophila of the first population is produced, and a fourth population of Drosophila comprising the male Drosophila of the second population is produced; crossing the third and fourth population of Drosophila to produce a fifth population of Drosophila comprising male and female Drosophila; and selecting female Drosophila comprising the heterologous gene of interest from the fifth population of Drosophila.

In one embodiment, the regulatable promoter is selected from the group consisting of heat shock promoter, Gal40, Gal80, Tet, and RU486.

In another embodiment, the male and female Drosophila of the first population further comprises a sequence encoding yeast Gal4 In another embodiment, the male and female Drosophila of the second population further comprises an upstream activator sequence operably linked to the heterologous gene of interest

In another embodiment, the insects of the fifth population are Drosophila embryos.

In another embodiment, the female Drosophila of the fifth population express the fluorescent protein.

In another embodiment, the step selecting female Drosophila comprising the heterologous gene of interest from the fifth population of insects comprises selecting female Drosophila which express the fluorescent protein.

In another embodiment, the step selecting female Drosophila comprising the heterologous gene of interest from the fifth population of insects comprises selecting Drosophila insects using flow cytometry.

In another embodiment, the flow cytometry is performed using a complex object parametric analyzer and sorter In another embodiment, the pro-apoptotic gene is selected from the group consisting of head involution defective, reaper, grim, hid-ala, ICE, and ced-3.

In another embodiment, the sequence encoding a fluorescent protein encodes a green fluorescent protein.

In another embodiment, the heterologous gene of interest is a neurodegenerative disease gene.

In another embodiment, the Drosophila of the second population further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the fifth population of Drosophila is placed in contact with rearing media comprising one or more test compounds.

The invention also encompasses a method for producing a population of female insects comprising a heterologous gene of interest, comprising: preparing a first population of insects comprising male and female insects wherein the male insects comprise a pro-apoptotic gene operably linked to a regulatable promoter integrated into the Y chromosome, and wherein the male and female insects further comprises a sequence encoding yeast Gal4; preparing a second population of insects comprising male and female insects wherein the female insects comprises an attached-X chromosome, and wherein a pro-apoptotic gene operably linked to a regulatable promoter is integrated into the attached-X chromosome, and wherein the male and female insects further comprises an upstream activator sequence operably linked to a heterologous gene of interest, and wherein the male insects of the second population comprise a sequence encoding a fluorescent protein integrated into the X chromosome; inducing the regulatable promoter of the first and second populations such that a third population of insects comprising the female insects of the first population is produced, and a fourth population of insects comprising the male insects of the second population is produced; crossing the third and fourth population of insects to produce a fifth population of insects comprising male and female insects; and selecting female insects comprising the heterologous gene of interest from the fifth population of insects.

In one embodiment, the regulatable promoter is selected from the group consisting of heat shock promoter, Gal40, Gal80, Tet, and RU486.

In another embodiment, the insects of the fifth population are insect embryos.

In another embodiment, the female insects of the fifth population express the fluorescent protein.

In another embodiment, the step selecting female insects comprising the heterologous gene of interest from the fifth population of insects comprises selecting female insects which express the fluorescent protein.

In another embodiment, the step of selecting female insects comprising the heterologous gene of interest from the fifth population of insects comprises selecting female insects using flow cytometry.

In another embodiment, the flow cytometry is performed using a complex object parametric analyzer and sorter.

In another embodiment, the insects are selected from the group consisting of Drosophila, silkworm, and mosquito.

In another embodiment, the pro-apoptotic gene is selected from the group consisting of head involution defective, reaper, grim, hid-ala, ICE, and ced-3.

In another embodiment, the sequence encoding a fluorescent protein encodes a green fluorescent protein.

In another embodiment, the heterologous gene of interest is a neurodegenerative disease gene.

In another embodiment, the insects of the second population further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the fifth population of insects is placed in contact with rearing media comprising one or more test compounds.

The invention also encompasses a method for producing a population of female Drosophila comprising a heterologous gene of interest, comprising: preparing a first population of Drosophila comprising male and female Drosophila wherein each of the male Drosophila comprise a pro-apoptotic gene operably linked to a regulatable promoter integrated into the Y chromosome, and wherein each of the male and female Drosophila further comprises a sequence encoding yeast Gal4; preparing a second population of Drosophila comprising male and female Drosophila wherein each of the female Drosophila comprises an attached-X chromosome, and wherein a pro-apoptotic gene operably linked to a regulatable promoter is integrated into the attached-X chromosome, and wherein each of the male and female Drosophila further comprises an upstream activator sequence operably linked to a heterologous gene of interest, and wherein the male Drosophila of the second population comprise a sequence encoding a fluorescent protein integrated into the X chromosome; inducing the regulatable promoter in the first and second populations of Drosophila such that a third population of Drosophila comprising the female Drosophila of the first population is produced, and a fourth population of Drosophila comprising the male Drosophila of the second population is produced; crossing the third and fourth population of Drosophila to produce a fifth population of Drosophila comprising male and female Drosophila; and selecting female Drosophila comprising the heterologous gene of interest from the fifth population of Drosophila.

In one embodiment, the regulatable promoter is selected from the group consisting of heat shock promoter, Gal40, Gal80, Tet, and RU486.

In another embodiment, the insects of the fifth population are Drosophila embryos.

In another embodiment, the female Drosophila of the fifth population express the fluorescent protein.

In another embodiment, the step of selecting female Drosophila comprising the heterologous gene of interest from the fifth population of Drosophila comprises selecting female Drosophila which express the fluorescent protein.

In another embodiment, the step of selecting female Drosophila comprising the heterologous gene of interest from the fifth population of Drosophila comprises selecting Drosophila using flow cytometry.

In another embodiment, the flow cytometry is performed using a complex object parametric analyzer and sorter In another embodiment, the pro-apoptotic gene is selected from the group consisting of head involution defective, reaper, grim, hid-ala, ICE, and ced-3.

In another embodiment, the sequence encoding a fluorescent protein encodes a green fluorescent protein.

In another embodiment, the heterologous gene of interest is a neurodegenerative disease gene.

In another embodiment, the Drosophila of the second population further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

In another embodiment, the fifth population of Drosophila is placed in contact with rearing media comprising one or more test compounds.

The invention also encompasses a method for producing a population of male insects, comprising: preparing a first population of insects comprising male and female insects wherein each of the male insects comprise a pro-apoptotic gene operably linked to a regulatable promoter integrated into the Y chromosome; preparing a second population of insects comprising male and female insects wherein each of the male insects comprises a sequence encoding a fluorescent protein integrated into the Y chromosome; inducing the regulatable promoter in the first population such that a third population of insects comprising the female insects of the first population is produced; selecting male insects from the second population which express the fluorescent protein such that a fourth population of insects comprising the male insects of the second population is produced; crossing the third and fourth population of insects to produce a fifth population of insects comprising male and female insects; and selecting male insects from the fifth population of insects.

In one embodiment, the regulatable promoter is selected from the group consisting of heat shock promoter, Gal40, Gal80, Tet, and RU486.

In one embodiment, the male insects of the second population which express the fluorescent protein are selected using flow cytometry.

In another embodiment, the flow cytometry is performed using a complex object parametric analyzer and sorter

The invention also encompasses a method for producing a population of male insects, comprising: preparing a first population of insects comprising male and female insects wherein each of the male insects comprise a pro-apoptotic gene operably linked to a regulatable promoter integrated into the Y chromosome; preparing a second population of insects comprising male and female insects wherein each of the female insects comprises an attached-X chromosome, and wherein a pro-apoptotic gene operably linked to a regulatable promoter is integrated into the attached-X chromosome, and wherein the second population of insects further comprise a sequence encoding a fluorescent protein integrated into the Y chromosome; inducing the regulatable promoter in each of the first and second populations such that a third population of insects comprising the female insects of the first population is produced, and a fourth population of insects comprising the male insects of the second population is produced; crossing the third and fourth population of insects to produce a fifth population of insects comprising male and female insects; and selecting male insects from the fifth population of insects.

The invention also encompasses a method for producing a population of male insects comprising a heterologous gene of interest, comprising: preparing a first population of insects comprising male and female insects wherein each of the male insects comprise a pro-apoptotic gene operably linked to a regulatable promoter integrated into the Y chromosome, wherein each of the male and female insects further comprises a sequence encoding yeast Gal4; preparing a second population of insects comprising male and female insects wherein each of the male insects comprises a sequence encoding a fluorescent protein integrated into the Y chromosome, and wherein each of the male and female insects further comprises an upstream activator sequence operably linked to a heterologous gene of interest; inducing the regulatable promoter in the first population such that a third population of insects comprising the female insects of the first population is produced; selecting male insects from the second population which express the fluorescent protein such that a fourth population of insects comprising the male insects of the second population is produced; crossing the third and fourth population of insects to produce a fifth population of insects comprising male and female insects; and selecting male insects comprising the heterologous gene of interest from the fifth population of insects.

In one embodiment, the regulatable promoter is selected from the group consisting of heat shock promoter, Gal40, Gal80, Tet, and RU486.

In another embodiment, the insects of the fifth population are insect embryos.

The method of claim 127, wherein the male insects of the fifth population express the fluorescent protein.

In another embodiment, the step of selecting male insects comprising the heterologous gene of interest from the fifth population of insects comprises selecting male insects which express the fluorescent protein.

In another embodiment, the step of selecting male insects comprising the heterologous gene of interest from the fifth population of insects comprises selecting male insects using flow cytometry.

In another embodiment, the flow cytometry is performed using a complex object parametric analyzer and sorter

In another embodiment, the insects are selected from the group consisting of Drosophila, silkworm, and mosquito.

In another embodiment, the pro-apoptotic gene is selected from the group consisting of head involution defective, reaper, grim, hid-ala, ICE, and ced-3.

In another embodiment, the sequence encoding a fluorescent protein encodes a green fluorescent protein.

In another embodiment, the heterologous gene of interest is a neurodegenerative disease gene.

In one embodiment, the male insects of the second population which express the fluorescent protein are selected using flow cytometry.

In another embodiment, the flow cytometry is performed using a complex object parametric analyzer and sorter

The invention also encompasses a method for producing a population of male insects comprising a heterologous gene of interest, comprising: preparing a first population of insects comprising male and female insects wherein each of the male insects comprise a pro-apoptotic gene operably linked to a regulatable promoter integrated into the Y chromosome, wherein each of the male and female insects further comprises a sequence encoding yeast Gal4; preparing a second population of insects comprising male and female insects wherein each of the female insects comprises an attached-X chromosome, and wherein a pro-apoptotic gene operably linked to a regulatable promoter is integrated into the attached-X chromosome, and wherein each of the male and female insects further comprises an upstream activator sequence operably linked to a heterologous gene of interest, and wherein the second population of insects further comprise a sequence encoding a fluorescent protein integrated into the Y chromosome: inducing the regulatable promoter in the first and second populations such that a third population of insects comprising the female insects of the first population is produced, and a fourth population of insects comprising the male insects of the second population is produced; crossing the third and fourth population of insects to produce a fifth population of insects comprising male and female insects; and selecting male insects comprising the heterologous gene of interest from the fifth population of insects.

The invention also encompasses a method for producing a humanized population of female insects, comprising: preparing a first population of insects comprising male and female insects wherein each of the male insects comprise a pro-apoptotic gene operably linked to a regulatable promoter integrated into the Y chromosome, and wherein each of the male and female insects further comprises a sequence encoding yeast Gal4; preparing a second population of insects comprising male and female insects wherein each of the female insects comprises an attached-X chromosome, and wherein a pro-apoptotic gene operably linked to a regulatable promoter is integrated into the attached-X chromosome, and wherein each of the male and female insects further comprises an upstream activator sequence operably linked to a human gene of interest, and wherein the male insects of the second population comprise a sequence encoding a fluorescent protein integrated into the X chromosome; inducing the regulatable promoter in the first and second populations such that a third population of insects comprising the female insects of the first population is produced, and a fourth population of insects comprising the male insects of the second population is produced; crossing the third and fourth population of insects to produce a fifth population of insects comprising male and female insects; and selecting humanized female insects comprising the human gene of interest from the fifth population of insects.

In one embodiment, the regulatable promoter is selected from the group consisting of heat shock promoter, Gal40, Gal80, Tet, and RU486.

In another embodiment, the insects of the fifth population are insect embryos.

In another embodiment, the female insects of the fifth population express the fluorescent protein.

In another embodiment, the step of selecting humanized female insects comprising the human gene of interest from the fifth population of insects comprises selecting female insects which express the fluorescent protein.

In another embodiment, the step of selecting humanized female insects comprising the human gene of interest from the fifth population of insects comprises selecting female insects using flow cytometry.

In another embodiment, the flow cytometry is performed using a complex object parametric analyzer and sorter In another embodiment, the insects are selected from the group consisting of Drosophila, silkworm, and mosquito.

In another embodiment, the pro-apoptotic gene is selected from the group consisting of head involution defective, reaper, grim, hid-ala, ICE, and ced-3.

In another embodiment, the sequence encoding a fluorescent protein encodes a green fluorescent protein.

In another embodiment, the human gene of interest is a neurodegenerative disease gene.

In another embodiment, the insects of the second population further comprise, integrated into the X chromosome, a female sterile mutation.

In another embodiment, the female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

The invention also encompasses a method for producing a humanized population of male insects, comprising: preparing a first population of insects comprising male and female insects wherein each of the male insects comprise a pro-apoptotic gene operably linked to a regulatable promoter integrated into the Y chromosome, and wherein each of the male and female insects further comprises a sequence encoding yeast Gal4; preparing a second population of insects comprising male and female insects wherein each of the male insects comprises a sequence encoding a fluorescent protein integrated into the Y chromosome, and wherein each of the male and female insects further comprises an upstream activator sequence operably linked to a human gene of interest; inducing the regulatable promoter in the first population such that a third population of insects comprising the female insects of the first population is produced; selecting from the second population, male insects which express the fluorescent protein such that a fourth population of insects comprising the male insects of the second population is produced; crossing the third and fourth population of insects to produce a fifth population of insects comprising male and female insects; and selecting humanized male insects comprising the human gene of interest from the fifth population of insects.

In one embodiment, the regulatable promoter is selected from the group consisting of heat shock promoter, Gal40, Gal80, Tet, and RU486.

In another embodiment, the insects of the fifth population are insect embryos.

In another embodiment, the male insects of the fifth population express the fluorescent protein.

In another embodiment, the step selecting humanized male insects comprising the human gene of interest from the fifth population of insects comprises selecting male insects which express the fluorescent protein.

In another embodiment, the step of selecting humanized male insects comprising the human gene of interest from the fifth population of insects comprises selecting male insects using flow cytometry.

In another embodiment, the flow cytometry is performed using a complex object parametric analyzer and sorter

In another embodiment, the insects are selected from the group consisting of Drosophila, silkworm, and mosquito.

In another embodiment, the pro-apoptotic gene is selected from the group consisting of head involution defective, reaper, grim, hid-ala, ICE, and ced-3.

In another embodiment, the sequence encoding a fluorescent protein encodes a green fluorescent protein.

In another embodiment, the human gene of interest is a neurodegenerative disease gene.

In one embodiment, the male insects of the second population which express the fluorescent protein are selected using flow cytometry.

In another embodiment, the flow cytometry is performed using a complex object parametric analyzer and sorter

The invention also encompasses a method for producing a humanized population of male insects, comprising: preparing a first population of insects comprising male and female insects wherein each of the male insects comprise a pro-apoptotic gene operably linked to a regulatable promoter integrated into the Y chromosome, and wherein each of the male and female insects further comprises a sequence encoding yeast Gal4; preparing a second population of insects comprising male and female insects wherein each of the female insects comprises an attached-X chromosome, and wherein a pro-apoptotic gene operably linked to a regulatable promoter is integrated into the attached-X chromosome, and wherein each of the male and female insects further comprises an upstream activator sequence operably linked to a human gene of interest, and wherein the second population of insects further comprise a sequence encoding a fluorescent protein integrated into the Y chromosome; inducing the regulatable promoter in the first and second populations such that a third population of insects comprising the female insects of the first population is produced, and a fourth population of insects comprising the male insects of the second population is produced; crossing the third and fourth population of insects to produce a fifth population of insects comprising male and female insects; and selecting humanized male insects comprising the human gene of interest from the fifth population of insects.

The invention also encompasses a male non-human animal comprising a pro-apoptotic gene operably linked to a regulatable promoter integrated into the Y chromosome, wherein the regulatable promoter is not a heat-shock promoter.

The invention also encompasses a male non-human animal comprising a pro-apoptotic gene operably linked to a regulatable promoter integrated into the Y chromosome, and further comprising integrated into its genome, a nucleic acid sequence encoding Gal4 operably linked to a neuronal or glial-specific promoter.

The invention also encompasses a male insect comprising a pro-apoptotic gene operably linked to a regulatable promoter integrated into the Y chromosome, and further comprises, integrated into the genome of the male insect a nucleic acid sequence encoding Gal4 operably linked to a neuronal or glial-specific promoter.

The invention also encompasses a male Drosophila comprising a pro-apoptotic gene operably linked to a regulatable promoter integrated into the Y chromosome and further comprises, integrated into the genome of the male Drosophila a nucleic acid sequence encoding Gal4 operably linked to a neuronal or glial-specific promoter

The invention also encompasses a female non-human animal comprising an attached-X chromosome, and wherein a pro-apoptotic gene is integrated into the attached-X chromosome.

The invention also encompasses a female non-human animal comprising an attached-X chromosome, wherein a pro-apoptotic gene is integrated into the attached-X chromosome, and wherein the female animal further comprises an upstream activator sequence operably linked to a heterologous gene of interest.

The invention also encompasses a population of female non-human animals comprising an attached-X chromosome, wherein a pro-apoptotic gene is integrated into the attached-X chromosome, and wherein the female animal further comprises an upstream activator sequence operably linked to a heterologous gene of interest, and further comprises a sequence encoding a fluorescent protein integrated into a sex chromosome.

The invention also encompasses a female non-human animal comprising an attached-X chromosome and wherein a pro-apoptotic gene is integrated into the attached-X chromosome, and wherein the female animal further comprises an upstream activator sequence operably linked to a heterologous gene of interest and wherein the female animal further comprises a sequence encoding a fluorescent protein integrated into a sex chromosome and wherein the female animal further comprises, integrated into the X chromosome, a female sterile mutation.

The invention also encompasses a female insect comprising an attached-X chromosome, wherein a pro-apoptotic gene is integrated into the attached-X chromosome.

The invention also encompasses a female Drosophila comprising an attached-X chromosome, and wherein a pro-apoptotic gene is integrated into the attached-X chromosome.

### BRIEF DESCRIPTION OF THE FIGURES

FIG. 1 shows a schematic of the genetics of the methods of the invention for the generation of sorted single sex Drosophila embryos.

FIG. 2 shows a variation of the scheme shown in FIG. 1.

FIG. 3 shows a variation of the scheme shown in FIG. 1.

FIG. 4 shows a variation of the scheme shown in FIG. 1.

FIG. 5 shows a variation of the scheme shown in FIG. 1.

### DETAILED DESCRIPTION OF THE INVENTION

The present invention provides a method for the preparation of single sex populations of organisms, preferably Drosophila, by utilizing inducible pro-apoptotic genes (which cause cell death) in a sex-specific manner. The invention, more specifically relates to the sex-specific sorting of non-human animal embryos, preferably insect embryos, utilizing flow cytometry and fluorescent labels which are expressed exclusively in the particular sexed insect of interest. Moreover, the invention provides a method for evaluating test compounds for their ability to act on a particular disease, preferably a neurodegenerative disease, wherein the compound is evaluated in animals which have been sex-sorted according to the methods of the invention.

Definitions

As used herein, the term “pro-apoptotic gene” refers to a gene, the expression of which controls and/or executes apoptosis, or programmed cell death, and further refers to genes which are associated with apoptosis. Apoptosis is a prominent feature of normal development throughout the animal kingdom, and occurs in a morphologically characteristic and reproducible manner. During apoptosis, the cell cytoplasm and nucleus of the cell condense, while the morphology of cellular organelles remains essentially intact. The cell fragments and is eventually engulfed by phagocytic cells. It is understood that apoptosis is the result of an active cellular program, and it is thought that the activity of certain “pro-apoptotic genes” is required for controlling and/or executing programmed cell death. A “pro-apoptotic gene” as used herein can refer to several genes including the head involution defective (hid), reaper, grim, hid/ala genes from Drosophila, the ced-3 gene from C. elegans, and the mammalian ice gene. A “pro-apoptotic gene” according to the invention also refers to homologs, and variants of the foregoing, provided that the homolog or variant is associated with apoptosis.

As used herein, a “regulatable promoter” refers to a promoter that is only expressed in the presence of an exogenous or endogenous chemical or stimulus (for example an alcohol, a hormone, or a growth factor), or in response to developmental changes, or at particular stages of differentiation, or in particular tissues or cells, or in response to a stimulus such as temperature. As used herein, a “regulatable promoter” refers preferably to a heat shock promoter which is expressed in response to a shift to an elevated temperature (more specifically, a temperature shift from about 18°-25° C. to at least 37° C. for at least 10-15 minutes). Examples of heat shock promoters useful in the invention include, but are not limited to, the promoters which regulate the expression of hsp70, hsp22, hsp23, hsp26, hsp27, hsp67b, hsp83, Hsc70-1, Hsc70-Z Hsc70-3, Hsc70-4, Hsc70-5, and Hsc70-6. A “regulatable promoter” as used herein refers to other genetic elements which are known to be useful for the regulation of gene expression including, but not limited to Gal80, Gal-ER, tet and Ru486 (as described in, for example, McGuire et al. (2004, SciSTKE 220:16), Brasselman et al. (1993, Proc. Natl. Acad. Sci. USA 90:1657), Gossen and Bujard (Proc. Natl. Acad. Sci. U.S.A. (1992) 89:5547, and Osterwalder et al. (2001, Proc. Natl. Acad. Sci. USA 23:12596), respectively).

As used herein the term “X-chromosome” refers to one of the heterologous sex chromosomes in animals such as mammals, insects and amphibians. As used herein the term “X-chromosome” is understood to include X-chromosome balancers which contain multiple inversions and/or translocations, and are utilized to minimize recombination events between homologous chromosomes. This provides a means of maintaining heterozygous stocks. The use of balancer chromosomes is known in the art, and a description may be found, for example, in Greenspan, R. J. (1997) Fly Pushing: The TheorEy and Practice of Drosophila Genetics, Cold Spring Harbor-Laboratory Press. As used herein, the term “sex chromosome” refers to the X or Y chromosome, or a balancer X or Y chromosome.

As used herein, the term “female sterile mutation” refers to a mutation in the X chromosome of a female organism which confers sterility to the female organism carrying the mutation. A “female sterile mutation” refers herein to a genetic mutation which prevents an organism carrying the mutation from being fertile. For example, a female animal that is homozygous for a recessive female sterile mutation, may be incapable of producing eggs but her progeny may subsequently die as embryos or larvae. A number of “female sterile mutations” are known to those of skill in the art and include, but are not limited to, fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll} and fs(1)pcx (See, e.g., Perrimon et al., 1984 Genetics, 108:559).

As used herein a “heterologous gene” is a gene or gene fragment that encodes a protein which is obtained from one or more sources other than the genome of the organism within which it is ultimately expressed. The source can be natural, e.g., the gene can be obtained from another source of living matter, such as bacteria, virus, yeast, fungi, insect, human and the like. The source can also be synthetic, e.g., the gene or gene fragment can be prepared in vitro by chemical synthesis. A “heterologous gene” as used herein can refer to a gene which is derived form a different species that the organism in which it is ultimately expressed. A “heterologous gene” useful in the invention can be a human gene, can be a disease gene, or a human disease gene, and further can be a human neurodegenerative disease gene. “Heterologous” can also be used in situations where the source of the gene fragment is elsewhere (e.g., derived from a different locus) in the genome of the organism in which it is ultimately expressed.

As used herein, a “neurodegenerative disease gene” refers to a gene, the normal expression of which, the aberrant expression of which, or the mutation of which is associated with the occurrence of a neurodegenerative disease. A “neurodegenerative disease” as used herein refers to degenerative disorders affecting the central or peripheral nervous system (including both neuronal cells and/or glia), including, but not limited to Parkinson's disease, Alzheimer's disease, Huntington's disease, amyotrophic lateral sclerosis, epilepsy, Tourette's syndrome, stroke, ischemic brain injury, and traumatic brain injury. “Neurodegenerative disease” as used herein refers to one or more diseases including, but not limited to Parkinson's Disease, Alzheimer's Disease, Huntington's Disease, spinocerebellar ataxia (SCA), age-related memory impairment, agyrophilic grain dementia, Parkinsonism-dementia complex of Guam, auto-immune conditions (eg Guillain-Barre syndrome, Lupus), Biswanger's disease, brain and spinal tumors (including neurofibromatosis), cerebral amyloid angiopathies (Journal of Alzheimer's Disease vol 3, 65-73 (2001)), cerebral palsy, chronic fatigue syndrome, corticobasal degeneration, conditions due to developmental dysfunction of the CNS parenchyma, conditions due to developmental dysfunction of the cerebrovasculature, dementia-multi infarct, dementia-subcortical, dementia with Lewy bodies, dementia of human immunodeficiency virus (HIV), dementia lacking distinct histology, Dementia Pugilistica, diffues neurofibrillary tangles with calcification, diseases of the eye, ear and vestibular systems involving neurodegeneration (including macular degeneration and glaucoma), Down's syndrome, dyskinesias (Paroxysmal), dystonias, essential tremor, Fahr's syndrome, fronto-temporal dementia and Parkinsonism linked to chromosome 17 (FTDP-17), frontotemporal lobar degeneration, frontal lobe dementia, hepatic encephalopathy, hereditary spastic paraplegia, hydrocephalus, pseudotumor cerebri and other conditions involving CSF dysfunction, Gaucher's disease, Hallervorden-Spatz disease, Korsakoff's syndrome, mild cognitive impairment, monomelic amyotrophy, motor neuron diseases, multiple system atrophy, multiple sclerosis and other demyelinating conditions (eg leukodystrophies), myalgic encephalomyelitis, myoclonus, neurodegeneration induced by chemicals, drugs and toxins, neurological manifestations of AIDS including AIDS dementia, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, oculopharyngeal muscular dystrophy, neurological manifestations of Polio myelitis including non-paralytic polio and post-polio-syndrome, primary lateral sclerosis, prion diseases including Creutzfeldt-Jakob disease (including variant form), kuru, fatal familial insomnia, Gerstmann-Straussler-Scheinker disease and other transmissible spongiform encephalopathies, prion protein cerebral amyloid angiopathy, postencephalitic Parkinsonism, progressive muscular atrophy, progressive bulbar palsy, progressive subcortical gliosis, progressive supranuclear palsy, restless leg syndrome, Rett syndrome, Sandhoff disease, spasticity, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, vascular dementia, and Wilson disease. A “neurodegenerative disease gene” as used herein thus refers to one or more genes which are associated with the onset, maintenance, or pathology of one or more of the above diseases. “Neurodegenerative disease genes” of the invention are known to those of skill in the art, and include, but are not limited to presenilin 1, presenilin 2, nicastrin, APH-1a, APH-1b, PEN-2, Tau (and mutants and variants thereof, described below), AB42 [Wildtype], AB42 [Flemish mutation], AB42 [Italian mutation], AB42 [Arctic mutation], AB42 [Dutch mutation], AB42 [Iowa mutation], APP [Wildtype], APP [London mutation], APP [Swedish mutation], APP [French mutation], APP [German mutation], SirT 1-5, SCA1, huntington, alpha-synuclein, DJ-1, and PINK-1.

As used herein, the term “fluorescent protein” refers to any protein which fluoresces when excited with appropriate electromagnetic radiation. This includes proteins whose amino acid sequences are either natural or engineered. A “fluorescent protein” as used herein includes, but is not limited to any protein selected from the group consisting of green fluorescent protein (GFP), enhanced fluorescent proteins (including EGFP, ECFP (cyan fluorescent protein), and EYFP (yellow fluorescent protein)), reef coral fluorescent protein blue fluorescent protein, red fluorescent protein, DsRed and other engineered forms of GFP, including humanized or mutated fluorescent proteins.

As used herein, the term “upstream activator sequence” refers to a genetic sequence that is bound by a transcriptional activator, for example, the yeast transcriptional activator Gal4. An “upstream activator sequence” (UAS) used in the context of a Gal4 activated system is referred to herein as a Gal4/UAS system. In the Gal4/UAS system, a heterologous gene of interest is cloned into a construct downstream of a promoter bearing one or more copies of the Upstream Activator Sequence (UAS) bound by the yeast transcriptional activator Gal4. The transgene is introduced into an organism, e.g., an insect, by standard means (e.g., may be expressed as a transgene, or integrated into the insect chromosome). When one wishes to induce the heterologous gene of interest, the transgenic organisms comprising the heterologous gene operably linked to UAS are crossed with a strain that expresses the yeast Gal4 molecule, either generally or under control of a tissue- or developmental stage-specific, or chemically-, or environmentally-inducible promoter, such that the Gal4 activates the transcription of the UAS-linked transgene in those tissues where the Gal4 is expressed. This system can be used to achieve highly temporally, spatially restricted specific expression of a heterologous gene based on the ability to generate Gal4 transgenics that express the Gal4 in only limited tissues. In addition to Gal4/UAS, other transcriptional activators and their respective binding activation domains may be used according to the invention, and such activators are known to those of skill in the art.

As used herein, the term “non-human animal” refers to a non-human, multicellular organism having an embryonic or larval size of greater than 50 μm in diameter and preferably having at least one dimension ranging between 70 and 500 μm or larger. As used herein, a non-human animal is a multicellular organism having an embryonic or larval stage which can be contained within a single fluid droplet of at least 100 μm in diameter and up to 1 mm in diameter. “Non-human animals” of the invention include, but are not limited to animals of the phyla cnidaria, ctenophora, platyhelminthes, nematoda, annelida, mollusca, chelicerata, uniramia, crustacea and chordata. Uniramians include the subphylum hexapoda that includes insects such as the winged insects. Chordates include vertebrate groups such as mammals, birds, fish, reptiles and amphibians. Techniques for producing transgenic animals, which comprise the genetic elements described herein, that may be used in the method of the invention are well known in the art. A useful general textbook on this subject is Houdebine, Transgenic animals—Generation and Use (Harwood Academic, 1997)—an extensive review of the techniques used to generate transgenic animals.

As used herein, the term “phenotype” refers to an observable and/or measurable physical, behavioral, or biochemical characteristic of a non-human animal useful in the invention (e.g., a fly). The term “altered phenotype” as used herein, refers to a phenotype that has changed relative to the phenotype of a wild-type animal. Examples of altered phenotypes include a behavioral phenotype, such as appetite, mating behavior, and/or life span, that has changed by a measurable amount, e.g. by at least 10%, 20%, 30%, 40%, or more preferably 50%, relative to the phenotype of a control animal (wherein a “control animal” refers to an animal which does not express a heterologous gene of interest, or which has not been exposed to a candidate compound); or a morphological phenotype that has changed in an observable way, e.g. different growth rate of the animal; or different shape, size, color, or location of an organ or appendage; or different distribution, and/or characteristic of a tissue, as compared to the shape, size, color, location of organs or appendages, or distribution or characteristic of a tissue observed in a control animal. A “change in phenotype” or “change in altered phenotype”, or “difference in phenotype” as used herein, means a measurable and/or observable change in a phenotype relative to the phenotype of a control non-human animal, insect or fly. As used herein, the term “change in phenotype” or “difference in phenotype” further refers to an increase or decrease in the phenotypic characteristic of interest. Thus, where the phenotype is, for example, reduced or altered climbing behavior, an increase in the phenotype is a more exaggerated alteration in climbing, whereas a decrease in phenotype is a reduction in the severity of altered climbing (or an increase in climbing). Phenotypic traits which may be measured according to the invention include, but are not limited to those described in WO 04/006854, published Jan. 22, 2004 (incorporated herein in its entirety).

As used herein, the term “insect” refers to an organism classified in the class insecta, and preferably refers to an organism in the order diptera. Of particular use in many embodiments is an insect which is a fly. Examples of such flies include members of the phylum uniramians include the subphylum hexapoda that includes insects such as the winged insects, and preferably includes members of the family Drosophilidae, including Drosophila melanogaster. In certain embodiments, the flies are transgenic flies, e.g., transgenic Drosophila melanogaster. A transgenic animal is an animal comprising heterologous DNA (e.g., from a different species) incorporated into its chromosomes. In other embodiments, the animals contain a genetic alteration which results in a change in level of expression of an endogenous polypeptide (e.g., an alteration which produces a gain of function or a loss of function result). The term animal or transgenic animal can refer to animals at any stage of development, e.g. adult, fertilized eggs, embryos, larva, etc.

As used herein, the term “operably linked” refers to the respective coding sequence being fused to a promoter, enhancer, termination sequence, and the like, so that the coding sequence is faithfully transcribed, spliced, and translated, and the other structural features are able to perform their respective functions.

The present invention is based, in part, on the discovery that a pro-apoptotic gene may be used in conjunction with a regulatable promoter and chromosomally integrated fluorescent protein to permit the high throughput, automated sorting of single sex populations of non-human embryos and/or larvae (e.g., Drosophila larvae).

Generator Populations

The present invention utilizes two generator populations to produce single sex populations of non-human animals that can be subsequently mated and then sorted based on sex.

Female Generator Population

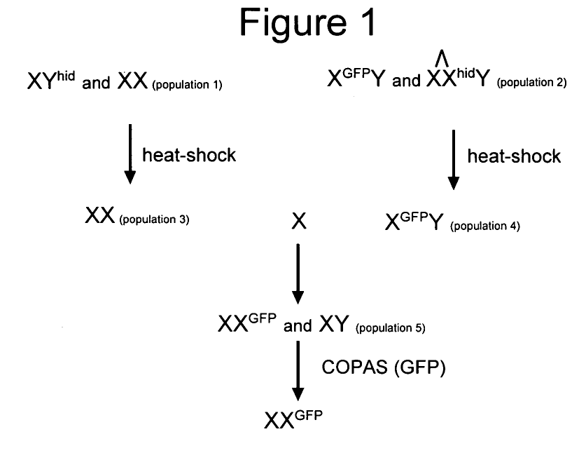

In a first embodiment, the invention provides a female generator population (population 1, FIGS. 1-5); that is, a mixed-sex population of non-human animals (e.g., Drosophila) which is used to produce a pure female population (population 3, FIGS. 1-5). All non-human animals known in the art for which the genetic manipulations described herein are possible may be used according to the invention. A non-human animal useful in the invention refers to a non-human, multicellular organism having an embryonic or larval size of greater than 50 μm in diameter and preferably having at least one dimension ranging between 70 and 500 μm or larger. As used herein, a non-human animal is a multicellular organism having an embryonic or larval stage which can be contained within a single fluid droplet of at least 100 μm in diameter and up to 1 mm in diameter. Preferred non-human animals of the invention are insects, nematodes, and amphibians. animals of the phyla cnidaria, ctenophora, platyhelminthes, nematoda, annelida, mollusca, chelicerata, uniramia, crustacea and chordata. Uniramians include the subphylum hexapoda that includes insects such as the winged insects. Chordates include vertebrate groups such as mammals, birds, fish, reptiles and amphibians. In a preferred embodiment, the animal is an insect. Methods for producing transgenic insects which may be used in the method of the invention are well known (see for example Loukeris et al.(1995), Science 270 2002-2005; and O'Brochta and Atkinson (1998) Scientific American 279 60-65).

More preferably a non-human animal of the invention is one or more Drosophila, silkworm, nematode, C. elegans, xenopus, zebrafish, zooplankton, medakafish, mosquito, and other flies.

The animals of the female generator population are genetically modified such that a pro-apoptotic gene, placed under the control of a regulatable promoter, is integrated into the Y chromosome, such that, when the regulatable promoter is activated, male animals will undergo apoptosis resulting in a pure population of female animals. Apoptosis is typically induced in the progeny of the female generator population such that the stock can be maintained, and exclusively virgin females can be isolated.

Methods for the integration of the regulatable promoter/pro-apoptotic gene into the Y sex chromosome are known in the art. Fly stocks which already contain, integrated into their genome the regulatable promoter/pro-apoptotic gene may be obtained from commercial sources such as the Bloomington stock center (Indiana University). Alternatively fly stocks may be generated using methods known in the art. Techniques which may be used to integrate the regulatable promoter/pro-apoptotic gene sequences into the Y chromosome may be found, for example, in Rubin and Spradling (1982), “Genetic Transformation of Drosophila with Transposable Element Vectors” Science, 218:348. Briefly, the method involves the ligation of DNAs of interest (e.g., regulatable promoter/pro-apoptotic gene) into an internally deleted P element. That is, a P element that lacks endogenous transposase activity but has retained its terminal repeats. Then by co-injecting the targeting vector with a secondary ‘helper’ P element (that has active transposase, but disrupted terminal repeats), the stable integration of the targeting vector and its associated DNA of interest but not the helper element can be achieved. The methods taught in Rubin and Spradling may be readily modified by one of skill in the art as needed to permit the generation of the Drosophila lines described herein.

Pro-apoptotic Genes

In one embodiment of the invention, the male animals of the female generator population are modified such that they contain integrated into the Y chromosome, a pro-apoptotic gene operably linked to a regulatable promoter. Pro-apoptotic genes according to the invention include any genes which are known in the art to induce or promote apoptosis in response to their expression. Thus, a pro-apoptotic gene useful in the invention refers to a gene, the expression of which controls and/or executes apoptosis, or programmed cell death, and further refers to genes which are associated with apoptosis. Apoptosis is a prominent feature of normal development throughout the animal kingdom, and occurs in a morphologically characteristic and reproducible manner. During apoptosis, the cytoplasm and nucleus of a cell condense, while the organelles' morphology remains essentially intact. Subsequently, the cell fragments and it is engulfed by phagocytic cells. It is understood that apoptosis is the result of an active cellular program, and it is thought that the activity of certain pro-apoptotic genes is required for controlling and/or executing programmed cell death. Pro-apoptotic genes useful in the present invention include, but are not limited to the head involution defective (hid) (Grether et al., 1995, Genes and Development 9:1694), reaper (White et al., 1994 Drosophila Science 264:677), grim, (Chen et al., 1996 Genes. Devel. 10:1773) hid/ala (Luque et al., 2002 Biochemistry 19:13663) genes from Drosophila, the ced-3 gene from C. elegans (Ellis et al., 1991 Annu. Rev. Cell biol. 7:663), and the mammalian ice gene (Whyte, 1996 Trends Cell Biol. 6:245). Pro-apoptotic genes useful according to the invention also include homologs, and variants of the foregoing, provided that the homolog or variant controls, executes, and/or is associated with apoptosis.

Regulatable Promoter

In one embodiment of the invention, a pro-apoptotic gene integrated into the Y chromosome of the female generator population is operably linked to a regulatable promoter. It will be understood by one of skill in the art that operable linkage between the regulatable promoter and pro-apoptotic gene means that activation of the promoter sequence (e.g., in response to whatever stimulus is appropriate to regulate the regulatable promoter) necessarily results in transcription of the pro-apoptotic gene.

Regulatable promoters useful in the invention include a promoter that is only expressed in the presence of an exogenous or endogenous chemical or stimulus (for example an alcohol, a hormone, or a growth factor), or in response to developmental changes, or at particular stages of differentiation, or in particular tissues or cells, or in response to a stimulus such as temperature. In a preferred embodiment, a regulatable promoter is a heat shock promoter which is expressed in response to a shift to an elevated temperature (generally, a temperature shift from about 18°-25° C. to a temperature of at least 37° C. for at least 10-15 minutes, and up to 2 hours or more). Examples of heat shock promoters useful in the invention include, but are not limited to, the promoters which regulate the expression of hsp70, hsp22, hsp23, hsp26, hsp27, hsp67b, hsp83, Hsc70-1, Hsc70-2, Hsc70-3, Hsc70-4, Hsc70-5, and Hsc70-6 (Ingolia and Craig, Nucleic Acids Res. 1980 8(19):4441-57; Arai et al., 1995 Japn J. Genetics 70:423).

In one embodiment of the invention, the regulatable promoter is not a heat shock promoter, but may be any other regulatable promoter described herein.

Other regulatable promoters include those that are controlled by the inducible binding, or activation, of a transcription factor, e.g., as described in U.S. Pat. Nos. 5,869,337 and 5,830,462 (incorporated herein by reference) by Crabtree et al., describing small molecule inducible gene expression (a genetic switch); International patent applications PCT/US94/01617, PCT/US95/10591, PCT/US96/09948 (incorporated herein by reference) and the like, as well as in other heterologous transcription systems such as those involving tetracyclin-based regulation reported by Bujard et al., generally referred to as an allosteric “off-switch” described by Gossen and Bujard (Proc. Natl. Acad. Sci. U.S.A. (1992) 89:5547) and in U.S. Pat. Nos. 5,464,758; 5,650,298; and 5,589,362 by Bujard et al. (incorporated herein by reference). Other regulatable transcription systems involve steroid or other hormone-based regulation. Other regulatable promoters which are known to those of skill in the art, including mutants, variants, and/or homologs may be likewise adapted according to the invention to regulate the expression of a pro-apoptotic gene of the invention.

A regulatable promoter useful in the invention includes other genetic elements which are known to be useful for the regulation of gene expression including, but not limited to Gal80, Gal-ER, tet and Ru486 (as described in, for example, McGuire et al. (2004, SciSTKE 220:16), Brasselman et al. (1993, Proc. Natl. Acad. Sci. USA 90:1657), Gossen and Bujard (Proc. Natl. Acad. Sci. U.S.A. (1992) 89:5547), Osterwalder et al. (2001, Proc. Natl. Acad. Sci. USA 23:12596), respectively). These are described in further detail below.

In a preferred embodiment, the regulatable promoter is a heat shock promoter and is operably linked to the Drosophila head involution defective gene. This combination is referred to herein as a hs-hid element. It will be understood by those of skill in the art that other combinations of pro-apoptotic genes and regulatable promoters may be used according to the invention in addition to the hs-hid element.

Male Generator Population

In a second embodiment, the invention provides a male generator population (i.e. population 2, FIG. 1-5); that is, a mixed-sex population of non-human animals (e.g., Drosophila) which is used to produce a pure male population (i.e. population 4, FIG. 1-5). All non-human animals known in the art for which the genetic manipulations described herein are possible may be used according to the invention. A non-human animal useful in the invention refers to a non-human, multicellular organism having an embryonic or larval size of greater than 50 μm in diameter and preferably having at least one dimension ranging between 70 and 500 μm or larger. As used herein, a non-human animal is a multicellular organism having an embryonic or larval stage which can be contained within a single fluid droplet of at least 100 μm in diameter and up to 1 mm in diameter. Preferred non-human animals of the invention are insects, nematodes, and amphibians. More preferably a non-human animal of the invention is one or more Drosophila, silkworm, nematode, C. elegans, xenopus, zebrafish, zooplankton, medakafish, mosquito, and other flies. Still more preferably, a non-human animal of the invention is a Drosophila.

The male generator population of the invention takes advantage of a genetic phenomenon referred to as the attached-X chromosome. In organisms wherein the X chromosome is telocentric (e.g., Drosophila), in some cases, two X chromosomes can become linked in the centromeric region such that they function as a single metacentric chromosome during meiosis. Because triple-X organisms show low viability (and any escapers are infertile), the only successful progeny of mating of an attached X animal with a normal male will be XˆXY female progeny and XY males. That is, the attached-X chromosome sorts exclusively from female parents to their daughters, and the X chromosome sorts exclusively from the male parents to their sons (e.g., flies). Taking advantage of this aberrant inheritance pattern, the present invention utilizes the attached-X chromosome by integrating a pro-apoptotic gene therein that is operably linked to a regulatable promoter. Thus, as noted above, the regulatable pro-apoptotic gene will only be carried by female animals on the XAX chromosome, and will not be present in male animals. Then, by activating the pro-apoptotic gene in the male generator stock, females are eliminated and only males are recovered. The activation of the pro-apoptotic gene is typically carried out in the progeny of the male generator stock such that the stock can be perpetuated.

Pro-apoptotic Genes and Regulatable Promoters

The pro-apoptotic genes and regulatable promoters that may be used in the male generator population are the same as those that may be utilized in the female generator population as described above. In a preferred embodiment, however, the pro-apoptotic gene is the hid gene that is operably linked to a regulatable heat shock promoter (i.e., the hs-hid element).

Female Sterile Mutations

In one embodiment of the invention, the male generator population further comprises, integrated into the X-chromosome (e.g., contained in the male generator animals), a recessive female sterile mutation. Attendant to the use of the attached-X genotype for the male generator population is the occurrence of non-dysjunction events in males, leading to the production of females which are fertile and not attached-X. The occurrence of such non-dysjunction events is rare, and takes place with a frequency of only 1/2000 to 1/5000. Regardless however, the rare occurrence of such a fertile female in the male generator population would impair the ability to produce a pure male population as these females would not carry the pro-apoptotic gene. Thus, in one embodiment, a recessive female sterile mutation is integrated into the X-chromosome, such that in any non-dysjunction event in males, the X chromosome carrying the recessive female sterile mutation will be segregated to the Fl female animals ensuring that females that arise from non-dysjunction events are homozygous for the recessive female sterile mutations and thus infertile and cannot contaminate the population. Female sterile mutations which may be used according to the invention include, but are not limited to the fs(1)K10 mutation, gastrulation defective mutation, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx (See, e.g., Perrimon et al., 1984 Genetics, 108:559).

The female sterile mutation may be integrated into the X chromosome using methods that are known in the art. Starting stocks of flies comprising sterile mutations may be obtained from commercial sources such as the Bloomington Stock Center (Indiana University), and X-linked female sterile mutations may be inserted into the chromosome using methods known in the art (see, e.g., Rubin and Spradling, supra).

Fluorescent Proteins

According to the invention, as described further below, the male and female generator populations are utilized to generate pure, single-sex populations of animals (e.g., Drosophila) which may then be crossed and sorted subsequently based on sex. The sorting event, in one embodiment, utilizes a fluorescent or other marker protein that is encoded by a sequence integrated into a sex chromosome of the animals of the male generator population, and which is placed under the control of a constitutive, tissue-specific, or regulatable promoter. Preferably, the promoter is a constitutive promoter. The specific sex chromosome into which the sequence encoding a fluorescent protein is integrated is determined by the specific sex of animal which is to be sorted (either positive or negative sorting). For example, if one of skill in the art wanted to sort female (FIG. 1) animals, then a sequence encoding a fluorescent protein would be integrated into the wild-type X chromosome of the male generator population. Because of the nature of the inheritance of attached-X chromosomes in the male generator population, as discussed above, the fluorescently labeled X chromosome will only be present in male animals. Thus, following the induction of the regulatable promoter in the male generator population, and the subsequent recovery of X^{GFP}Y males, a subsequent cross with normal XX females will yield progeny in which the labeled X chromosome is carried only by female offspring. Methods for integrating a sequence encoding a fluorescent protein into the X or Y chromosome of the male generator population are known in the art, and may be readily adapted from the general teachings of Rubin and Spradling described above.

Fluorescent proteins useful in the present invention include any protein that fluoresces when excited with appropriate electromagnetic radiation. This includes proteins whose amino acid sequences are either natural or engineered, or a combination thereof, such as mutant fluorescent proteins, which are based on a naturally occurring protein, but which have been modified, for example, to have a specific spectral shift, or other fluorescent characteristic. A fluorescent protein as used herein includes, but is not limited to any protein selected from the group consisting of green fluorescent protein (GFP), enhanced fluorescent proteins (including EGFP, ECFP (cyan fluorescent protein), and EYFP (yellow fluorescent protein)), reef coral fluorescent protein blue fluorescent protein, red fluorescent protein, DsRed and other engineered forms of GFP, including humanized or mutated fluorescent proteins. Fluorescent proteins useful in the present invention may be obtained from several commercial sources including, but not limited to Molecular Probes, Inc. (Eugene, Oreg.). Information regarding sequences encoding fluorescent proteins may be found on the world wide web at molecularprobes.com.

It will be understood by those of skill in the art that, in addition to the fluorescent markers described herein, other detectable markers known in the art may be employed in the present invention. Other detectable markers suitable for use in the present invention include any composition detectable by spectroscopic, photochemical, biochemical, immunochemical, electrical, optical or chemical means. Useful labels in the present invention include biotin for staining with labeled streptavidin conjugate, enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label. Other detection methods of particular use in the present invention include flow cytometry which is described in more detail below.

Heterologous Genes

In one embodiment of the invention, the male and female generator populations are genetically modified such that the offspring of the pure male and female populations which result therefrom, when crossed, will express a heterologous gene of interest, preferably in a tissue specific manner. There are many gene expression systems known in the art which may be adapted for use in the present invention. A preferred example of an inducible gene expression system of the invention is the Gal4/UAS system. Gal4/UAS is a system for regulated expression of a heterologous gene of interest. In this system, the heterologous gene of interest is cloned into a construct downstream of a promoter bearing one or more copies of the Upstream Activator Sequence (UAS) which may be activated by the yeast transcriptional activator Gal4. The heterologous gene of interest is introduced into a non-human animal, e.g., an insect, by standard means. When one wishes to induce the heterologous gene of interest, the resulting transgenic organisms are crossed with a strain that expresses the yeast Gal4 molecule, either generally or under control of a tissue- or developmental stage-specific promoter, such that the Gal4 activates the transcription of the UAS-linked heterologous gene in those tissues where the Gal4 is expressed. This system is well-established as described in Brand and Perrimon (1993, Development 118:401-415) and Roth et al. (1998, Development 125:1049-1057), the teachings of which are incorporated herein in their entirety. In addition, libraries of tissue specific GAL4 transgenic fly lines are available on the world wide web at, for example fly-trap.org and flymap.lab.nig.ac.jp/˜dclust/getdb.html.

More specifically, the female generator population is genetically modified such that it contains, stably integrated into the genome, the Gal4 encoding sequence in such a manner that it is operably linked to either an endogenous promoter sequence that provides for expression in the cells of interest, or an exogenous promoter (e.g., heterologous promoter), which will likewise provide for expression only in the cells of interest, or in the case of a temporally regulatable promoter, at a time of interest. Promoters which may be used to drive the expression of Gal4 may be alternative forms of regulatable promoters known in the art, and described herein, including chemically or temperature sensitive promoters. In a preferred embodiment, the promoter element operably linked to the Gal4 coding sequence is a neuronal- or glial-specific promoter; that is a promoter which is activated selectively in neurons or glia. As used herein, “neurons” refers to any neuronal cell in either the central or peripheral nervous system, including, but not limited to sensory neurons, motor neurons, intemeurons, autonomic neurons, and neuronal progenitors or precursors. As used herein, “glia” refers to glial cells of the central and peripheral nervous system including, but not limited to astrocytes, oligodentrocytes, schwann cells, microglia, radial glia, and further includes aberrant glial structures such as a glioma or astrocytoma. Neuronal specific promoters which are useful in the invention include, but are not limited to ELAV, α-enolase, β-actin, tau promoter, p35 promoter, nestin promoter, the GABA promoter, Ddc, nervana, scaberous, ace-1, acr-5, aex-3, apl-1, alt-1, cat-1, cat-2, cch-1, cdh-3, ceh-2, ceh-2, ceh-6, ceh-10, ceh-14, ceh-17, ceh-23, ceh-28, ceh-24, ceh-36, che-1, che-3, cgk-1, cha-1, cnd-1, cod-5, daf-1, daf-4, daf-7, daf-19, dbl-1, des-2, deg-1, deg-3, del-1, eat-4, eat-16, ehs-1, egl-10, egl-17, egl-19, egl-2, egl-36, egl-5, egl-8, fax-1, flp-1, flp-1, flp-3, flp-5, flp-6, flp-8, flp-12, flp-15, flp-3, flr-4, gcy-10, gcy-12, gcy-32, gcy-33, gcy-5, gcy-6, gcy-7, gcy-8, ggr-1, ggr-2, ggr-3, glr-1, glr-5, glr-7, glt-1, goa-1, gpa-1, gpa-1, gpa-2, gpa-3, gpa-4, gpa-5, gpa-6, gpa-7, gpa-8, gpa-9, gpa-10, gpa-11, gpa-13, gpa-14, gpa-15, gpa-16, gpb-2, gsa-1, ham-2, her-1, ida-1, ina-1, lim-4, lim-6, lim-6, lim-7, lin-11, lin-4, lin-45, mab-18, mec-3, mec-7, mec-8, mec-9, mec-18, mgl-1, mgl-2, mig-1, mig-13, mus-1, ncs-1, nhr-22, nhr-38, nhr-79, nmr-1, ocr-1, ocr-2, odr-1, odr-2, odr-2, odr-10, odr-3, odr-3, odr-7, opt-3, osm-10, osm-3, osm-9, pag-3, pha-1, pin-2, rab-3, ric-19, sak-1, sdf-13, sek-1, sek-2, sgs-1, snb-1, snt-1, sra-1, sra-10, sra-11, sra-6, sra7, sra-9, srb-6, srg-2, srg-8, srd-1, sre-1, srg-13, sro-1, str-1, str-2, str-3, syn-2, tab-1, tax-2, tax-4, tig-2, tph-1, ttx-3, ttx-3, unc-3, unc-4, unc-5, unc-8, unc-8, unc-11, unc-17, unc-18, unc-25, unc-29, unc-30, unc-37, unc-40, unc-43, unc-47, unc-55, unc-64, unc-86, unc-97, unc-103, unc-115, unc-116, unc-119, unc-129, vab-7. Examples of glial-specific promoters which may be used in the invention include, but are not limited to, glial fibrilary acidic protein (GFAP), MH-1, GCM, Repo (reverse polarity), and dEEAT 1 & 2. In addition to the control of Gal4 expression using tissue or other regulatable promoters, other expression regulation elements are known in the art and may be used according to the invention. For example, the female generator population may be further modified to include integrated into its chromosomes, a genetic element selected from, but not limited to Gal80, Gal-ER, tet and Ru486 (as described in, for example, McGuire et al. (2004, SciSTKE 220:16), Braselmann et al. (1993, Proc. Natl. Acad. Sci. USA 90:1657), Gossen and Bujard (Proc. Natl. Acad. Sci. U.S.A. (1992) 89:5547, and Osterwalder et al. (2001, Proc. Natl. Acad. Sci. USA 23:12596), respectively). Each of these genetic elements can function in concert with Gal4 to either repress its activity, or to stimulate Gal4 activity (e.g., act as a transcription factor for Gal4, inducible by a chemical or environmental stimulus), wherein the repression of Gal4 is relieved by one or more chemical or environmental stimuli. For example, Gal80 is known to repress Gal4 activity at certain temperatures. Thus, by including Gal80 in the female generator population (wherein Gal4 is also integrated under the control of a, for example, tissue specific promoter) Gal4 induction can be delayed by keeping the population at a certain temperature until a time when Gal4 activation is desired. The population can then be exposed to a temperature at which Gal80 no longer represses Gal4, permitting the activation of the UAS and expression of the heterologous gene of interest. This combination of Gal4 and Gal4 repressor permits one of skill in the art to regulate both the spatial and temporal aspects of heterologous gene expression.

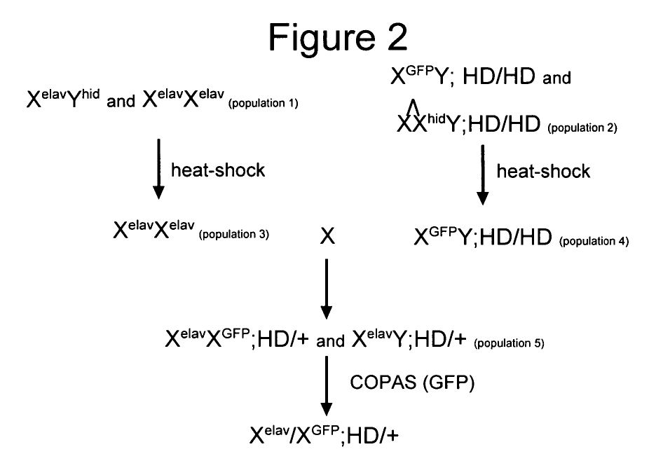

Similarly, the male generator population is genetically modified to include, stably integrated into the genome, an upstream activator sequence (UAS) operably linked to the heterologous gene of interest (e.g., FIGS. 2-5). Following the protocols described below, once the regulatable promoter which is operably linked to the pro-apoptotic gene is induced, the resulting pure male population will contain the UAS target construct (and the resulting pure female population will contain the Gal4 driver construct which is expressed preferably in a tissue specific manner).

Crossing the two single sex populations (populations 3 and 4 respectively in FIGS. 1-5) results in the induction of the heterologous gene of interest in the progeny, but expression of the gene is limited to the specific tissues (in the case of Gal4 under the control of a tissue specific promoter) in which Gal4 is expressed. For example, where Gal4 is under the control of a neuronal-specific promoter, the heterologous gene of interest will only be transactivated via the Gal4/UAS interaction in neuronal cells which express the Gal4.

The heterologous gene of the invention is a gene or gene fragment that encodes a protein and is obtained from one or more sources other than the genome of the organism within which it is ultimately expressed. The source can be natural, e.g., the gene can be obtained from another source of living matter, such as bacteria, virus, yeast, fungi, insect, human and the like. Preferably the heterologous gene is of human origin. The source can also be synthetic, e.g., the gene or gene fragment can be prepared in vitro by chemical synthesis. A heterologous gene can be a gene which is derived from a different species that the organism in which it is ultimately expressed. A heterologous gene useful in the invention can be a human gene, can be a disease gene, or a human disease gene, and further can be a human neurodegenerative disease gene. Heterologous genes can also be used in situations where the source of the gene fragment is elsewhere (e.g., derived from a different locus) in the genome of the organism in which it is ultimately expressed.

In one embodiment, the heterologous gene of interest is a disease gene; that is a gene which initiates, regulates, or maintains a given disease state in an animal. In a preferred embodiment, a heterologous gene useful in the invention is a neurodegenerative disease gene, preferably a human neurodegenerative disease gene. Neurodegenerative disease genes are known in the art and are described, for example in U.S. Application 20040076999, published Apr. 22, 2004 (the contents of which are incorporated herein by reference). Preferred neurodegenerative disease genes used in the invention include, but are not limited to presenilin 1, presenilin 2, nicastrin, APH-1a, APH-1b, PEN-2, Tau (and mutants and variants thereof, described below), AB42 [Wildtype], AB42 [Flemish mutation], AB42 [Italian mutation], AB42 [Arctic mutation], AB42 [Dutch mutation], AB42 [Iowa mutation], APP [Wildtype], APP [London mutation], APP [Swedish mutation], APP [French mutation], APP [German mutation], SirT 1-5, ataxin-1, huntingtin, alpha-synuclein, DJ-1, and PINK-1.

Based on the foregoing, the present invention thus provides populations of non-human animals which may be used to produce two single sex populations of non-human animals (populations 3 and 4 in FIG. 1) which contain, respectively, a Gal4 driver and UAS/heterologous gene-target. Thus, crossing populations 3 and 4 provides a convenient way to control the integration of the driver/target system in the genome of the resulting offspring, such that they express the heterologous gene of interest in a tissue-, time-, or stimulus-specific manner; that is, wherever and/or whenever Gal4 is expressed.

Sex-Specific Sorting Method

As previously described, the present invention provides a method for sex specific sorting of animals, more specifically embryos and/or larvae into single sex populations which may be used, for example, in screening assays to identify compounds or agents which modulate a phenotype. In one embodiment of the invention, the sex-specific sorting comprises two stages, the first being the sex-specific culling of one sex based on the sex-specific expression of a pro-apoptotic gene under the control of a regulatable promoter. The animal populations used in this first stage sort are described above as the female (first population) and male (second population) generator populations. The resulting pure female (third population) and pure male (fourth population) populations of animals are subsequently crossed to produce a mixed progeny population (fifth population). The second stage of sorting is then conducted based on the expression of a fluorescent protein or other marker, wherein the expression of the marker is restricted to a single sex of the fifth population. Single sex animals are selected from the fifth population that express the fluorescent marker, thus yielding a single sex population. As noted above, either pure male or pure female populations may be obtained in the second stage of sorting by varying which of the sex chromosomes the sequence encoding the fluorescent marker is integrated. For example, where a pure population of male animals (e.g., Drosophila) is desired, the sequence encoding the fluorescent protein is integrated, using methods known in the art and described herein, into the Y chromosome of the male generator population (second population). Conversely, where a pure female population is to be selected from the fifth population, the sequence encoding the fluorescent protein is integrated into the X chromosome of the male generator population (second population), and is thus only sorted to the female offspring contained within the fifth population.

Induction of the Pro-apoptotic Gene

Male and female generator strains as described herein include, integrated into the XˆX and Y chromosomes, respectively, a pro-apoptotic gene which is operably linked to a regulatable promoter. Accordingly, to induce apoptosis in the developing embryos of the male and female generator lines, the proper stimulus must be provided to induce the regulatable promoter to mediate transcription of the pro-apoptotic gene. As described above the regulatable promoter may be a promoter that is only expressed in the presence of an exogenous or endogenous chemical or stimulus (for example an alcohol, a hormone, or a growth factor), or in response to developmental changes, or at particular stages of differentiation, or in particular tissues or cells, or in response to a stimulus such as temperature. Thus, activation of the regulatable promoter requires that one of skill in the art provide the stimulus appropriate to the specific regulatable promoter used in the invention. For example, where the regulatable promoter is activated in response to a hormone, one of skill in the art would activate the regulatable promoter (and thus induce expression of the pro-apoptotic gene) by providing the animal expressing the modified sex chromosome (either Y or XˆX) with the appropriate hormone, in an appropriate concentration to activate the promoter.

In a preferred embodiment, a regulatable promoter is a heat shock promoter which is activated in response to an elevation in temperature (generally, a temperature shift from about 18°-25° C. to at least 37° C. for at least 10-15 minutes, and up to 2 hours or more). Examples of heat shock promoters useful in the invention include, but are not limited to, the promoters which regulate the expression of hsp70, hsp22, hsp23, hsp26, hsp27, hsp67b, hsp83, Hsc70-1, Hsc70-2, Hsc70-3, Hsc70-4, Hsc70-5, and Hsc70-6 (Ingolia and Craig, Nucleic Acids Res. 1980 8(19):4441-57; Arai et al., 1995 Japn J. Genetics 70:423). Methods for inducing heat shock promoters are understood in the art. In one embodiment of the present invention, the animals of the invention are Drosophila, and the following protocol may be used to induce the heat shock promoter, thus activating the chromosomally linked pro-apoptotic gene. Briefly, Drosophila embryos are collected on agar plates overnight (e.g., embryos derived from either the male or female generator population). This is considered day 0. Embryos are sieved through a sequential series of sieves (850 um, 425 um, and 125 um) in embryo wash solution comprising, for example, 0.7% NaCl and 0.03% Triton X-100, and are finally collected in a 50 ml conical tube. Embryos are gravity pelleted and resuspended in sterile inoculation solution comprising, for example 14.4% w/v sucrose, 0.7% w/v NaCl, and 0.05% Triton X-100. The embryos are then pipetted in solution into 8 oz stock bottles at a concentration of 600-700 embryos per bottle. This is considered day 1. The larvae are then heat shocked for 2 hours in a circulating water bath at 37° C. on day 3 and 4. The embryos which do not contain the hs-hid element should eclose on day 10.

The induction of the pro-apoptotic gene can be confirmed by detecting apoptosis in the animals (e.g., embryos and/or larvae) in which the gene is activated. Cells undergoing apoptosis show characteristic morphological and biochemical features. These features include chromatin aggregation, nuclear and cytoplasmic condensation, partition of cytoplasm and nucleus into membrane bound vesicles (apoptotic bodies) which contain ribosomes, morphologically intact mitochondria and nuclear material. In vivo, these apoptotic bodies are rapidly recognized and engulfed by either glia, macrophages, or adjacent epithelial cells. Due to this efficient mechanism for the removal of apoptotic cells in vivo no inflammatory response is elicited. Detection of any one or more of the foregoing is indicative of apoptosis in the embryos and/or larvae of the invention. Other morphological and biochemical aspects of apoptosis which may be detected so as to indicate the activation of a pro-apoptotic gene of the invention include, but are not limited to membrane blebbing, but no loss of integrity; aggregation of chromatin at the nuclear membrane; shrinking of cytoplasm and condensation of nucleus; fragmentation of cell into smaller bodies; formation of membrane bound vesicles (apoptotic bodies); mitochondria become leaky due to pore formation involving proteins of the bcl-2 family; energy (ATP)-dependent (active process, does not occur at 4° C.); non-random mono- and oligonucleosomal length fragmentation of DNA (Ladder pattern after agarose gel electrophoresis); prelytic DNA fragmentation; release of various factors (cytochrome C, AIF) into cytoplasm by mitochondria; activation of caspase cascade; alterations in membrane asymmetry (i.e., translocation of phosphatidyl-serine from the cytoplasmic to the extracellular side of the membrane).

Animal Crosses

Following activation of the pro-apoptotic genes in each of the male and female generator populations, populations of pure male or pure female animals are obtained, with the exception of the rare non-dysjunction event in the male generator population which is remedied by the inclusion of a female sterile mutation in the X chromosome of the male generator population. In one embodiment, following the generation of the third and fourth single sex populations, the single sex populations are bred to produce a mixed sex population. Methods of crossing various animals (e.g., Drosophila) are known in the art. Methods for crossing, and culturing Drosophila may be found, for example, in Ashbumer, In Drosophila melanogaster: A Laboratory Manual (1989); Greenspan, Cold Spring Harbor Laboratory Press, Fly Pushing: The Theory and Practice of Drosophila Genetics, Cold Spring Harbor Laboratory Press (1997).

As described above, following the crossing of the third and fourth populations of animals, same sex animals will be selected from the resulting fifth population based on the sex-specific expression of one or more markers; preferably one or more fluorescent proteins. A sequence encoding a fluorescent protein or other marker is integrated into one o the sex chromosomes of the male generator population such that in the resulting fifth population, only one sex will express the fluorescent protein. For example, if male animals, preferably male embryos or larvae are to be selected from the fifth population, then the sequence encoding the fluorescent protein is integrated into the Y chromosome, and if female animals, preferably female embryos or larvae, are to be selected from the fifth population, then the sequence encoding the fluorescent protein is integrated into the X chromosome. The Y linked fluorescent protein will segregate to only male animals or embryos/larvae thereof of the fifth population, and the X linked fluorescent protein will segregate to only female animals of embryos/larvae thereof in the fifth population.

Fluorescence-Based Sorting

The fifth population is sorted to select a single sex of animals from the mixed sex population. Sorting is performed based on the expression of a fluorescent marker exclusively in one sex or the other as described above.

Fluorescence detection can be performed using methods well known in the art. For example, fluorescent proteins useful in the present invention emit photons of light in response to excitation energy of the appropriate wavelength. Given fluorescent proteins have specific ranges of excitation spectra, and will likewise emit light at a certain range of emission spectra. One of skill in the art, based on the particular fluorescent protein used in the invention can excite the non-human animal, preferably embryos and/or larvae, with the appropriate excitation wavelength and detect the emission at the predicted wavelength using photon detectors and filters which are known in the art. In one embodiment, the fluorescently labeled non-human animal embryos and/or larvae are detected by flow cytometry. Embryos and/or larvae may then be sorted using fluorescent activated cell sorted (e.g., using a sorter available from Becton Dickinson Immunocytometry Systems, San Jose, Calif., USA; see also U.S. Pat. Nos. 5,627,037; 5,030,002; and 5,137,809). As embryos/larvae pass through the sorter, a laser beam excites the fluorescent compound while a detector determines whether a fluorescent compound is attached to the cell by detecting fluorescence, and subsequently sorts the embryos and/or larvae by deflecting embryos and/or larvae which express the fluorescent protein into one collection means, while deflecting embryos and/or larvae which do not express the fluorescent protein into a second collection means. Collection means useful in the invention include, but are not limited to a tube, culture well, flask, petri dish, and the like. Such a system can also count the number of embryos and/or larvae that are sorted, and additionally can quantitate the level of fluorescence.

In one embodiment, embryos and/or larvae derived from the fifth population of non-human animals are sorted using a complex object parametric analyzer and sorter (COPAS). COPAS-based sorting optically measures physical parameters including size, optical density, and the presence of fluorescent markers. Once analyzed, objects are sorted according to user selectable criteria, and then are dispensed into stationary receptacles (although the receptacles may be designed on a moveable platform or stage, such that different objects may be sorted into different receptacles). COPAS-based sorting methods are described in U.S. Pat. Nos. 6,657,713 and 6,400,453 (both of which are incorporated herein in their entirety), and an apparatus useful in the present invention for the performance of COPAS sorting of non-human animals, preferably embryos and/or larvae of the invention may be obtained from Union Biometrica, Somerville, Mass.

A schematic representation of the foregoing sorting method is shown in FIG. 1. Specifically, FIG. 1 shows a sorting method for producing a final population of female non-human animals. As can be seen in the figure, the female generator population (population 1) comprises male animals having integrated into the Y chromosome the pro-apoptotic gene hid which is under the control of a heat shock promoter. The male generator population (population 2) comprises the pro-apoptotic gene hid integrated into the attached-X chromosome (XAX) of the female animals, and further comprises a sequence encoding GFP integrated into the X chromosome. Each of populations 1 and 2 are subjected to heat shock as described above, thus producing the pure female population 3, and pure male population 4 comprising the X-linked GFP. Populations 3 and 4 are subsequently crossed to give rise to population 5. Population 5 is then subjected to COPAS sorting based on the female-specific expression of a fluorescent protein (in this scenario, GFP). It will be understood, based on the description provided herein, that the sorting method outlined in FIG. 1, can optionally include a female sterile mutation (e.g., fs(1)K10) integrated into the X chromosome of the male generator population. In addition the method of FIG. 1 can be modified to sort for male animals from population 5, by integrating a sequence encoding a fluorescent protein into the Y chromosome of the male generator population instead of the X chromosome.

The method shown in FIG. 1 can be modified further by including a heterologous gene expression system. FIG. 2 shows the sorting method of FIG. 1, modified such that the final sorted population expresses a heterologous gene of interest (e.g., a neurodegenerative disease gene). As shown in FIG. 2, the female generator population (population 1) comprises male animals having integrated into the Y chromosome the pro-apoptotic gene hid which is under the control of a heat shock promoter. The female generator population also includes a sequence encoding Gal4, the expression of which is directed by the neuronal specific promoter ELAV (the ELAV-Gal4 construct being designated as “elav” in the figure). The male generator population (population 2) comprises the pro-apoptotic gene hid integrated into the attached-X chromosome (XˆX) of the female animals, and further comprises a sequence encoding GFP integrated into the X chromosome. The designation HD/HD indicates that the male generator population also contains, integrated into its chromosomes, a UAS activator operably linked to a heterologous gene of interest, which in FIG. 2 is shown, for example, as the Huntington's disease gene (HD). Each of populations 1 and 2 are subjected to heat shock as described above, thus producing the pure female population 3, comprising the Gal4 driver, and pure male population 4 comprising the X-linked GFP, and UAS/HD target. Populations 3 and 4 are subsequently crossed to give rise to population 5. Population 5 is then subjected to COPAS sorting based on the female-specific expression of a fluorescent protein (in this scenario, GFP). It will be understood, based on the description provided herein, that, like the basis sorting method shown in FIG. 1, the sorting method outlined in FIG. 2, can optionally include a female sterile mutation (e.g., fs(1)K10) integrated into the X chromosome of the male generator population.

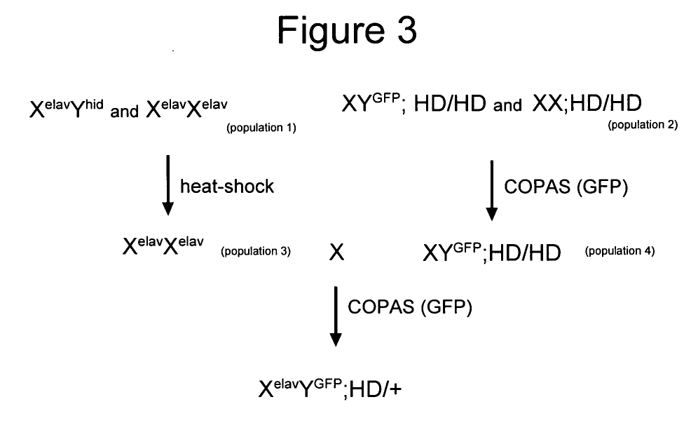

In addition to the primary sorting method described above and shown in FIG. 1, the present invention contemplates variations of this method that are nonetheless within the scope and spirit of the present invention. FIG. 3 shows a variation on the primary sorting method in which the male generator population is sorted to produce only male non-human animals by fluorescence-based sorting, rather than by integration and activation of a pro-apoptotic gene in the attached-X chromosome. As can be seen in FIG. 3, the male generator population is modified from that shown in FIG. 2, such that the female animals have the traditional XX genotype, and the male animals comprise a sequence encoding GFP (or other fluorescent protein) on the Y chromosome. An initial fluorescence based sorting step produces a pure population of male animals comprising GFP linked to the Y chromosome. The initial fluorescence-based sorting step can be carried out using COPAS as shown in FIG. 3, or may alternatively, employ other manual or automated methods known in the art for selecting male animals which express the fluorescent protein (e.g., flow cytometry, or manual “by hand” selection of fluorescent animals). The remainder of the sorting method shown in FIG. 3 is essentially the same as shown in FIG. 2, with populations 3 and 4 being crossed to produce mixed sex population 5 (not shown), from which is selected by COPAS (or other automated fluorescence-based selection method), male flies.

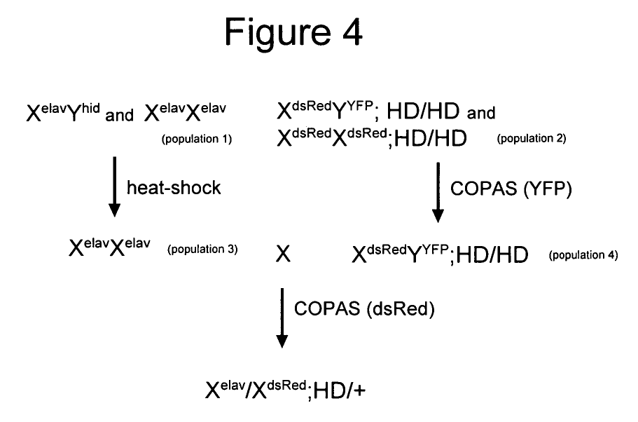

FIG. 4 shows a further variation on the basic selection method of FIG. 2 for the selection of female non-human animals. Again the male generator population is modified such that a sequence encoding a first fluorescent protein is integrated into the X chromosome of all animals in the population (dsRed in FIG. 4), and a sequence encoding a second fluorescent protein is integrated into the Y chromosome (yellow fluorescent protein; YFP in FIG. 4). The male generator population is subjected to fluorescence based sorting for the second fluorescent protein, thus producing a population (analogous to population 4 in FIG. 1) of pure males comprising dsRed integrated into the X chromosome and YFP integrated into the Y chromosome. The progeny of the subsequent cross of the pure female population resulting from the female generator population and the pure male population resulting from the male generator population are then subjected to fluorescence-based sorting using COPAS. This second COPAS sorting step takes advantage of the distinct fluorescent labels on each of the sex chromosomes of the pure male population such that the sorting step will sort positively for dsRed (that is, will select embryos/larvae which emit at the dsRed wavelength) and optionally will, in addition, sort negatively for YFP (that is, will reject, or sort into a waste population, embryos/larvae which emit at the YFP wavelength). This provides a mechanism for selecting against any male animals which may have been missed by the dsRed selection, thus producing a pure population of female animals.

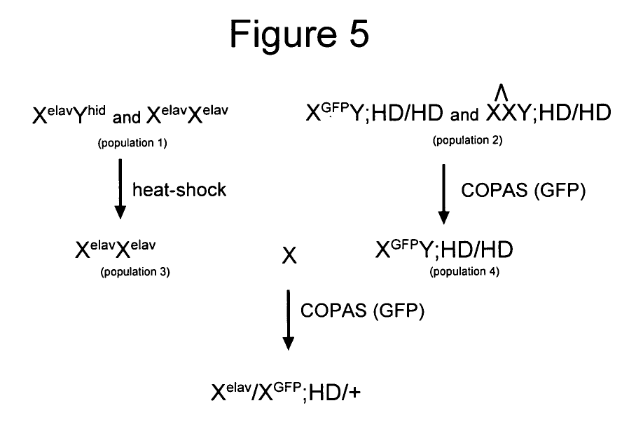

FIG. 5 shows yet another variation of the general sorting method shown in FIG. 2, in which female animals are selected. The method shown in FIG. 5 is essentially identical to that of FIG. 2, with the exception that there is no hs-hid element integrated into the attached-X chromosome of the male generator population. Instead, the male generator population is sorted into a pure male population by fluorescence based selection of male animals which have a sequence encoding a fluorescent protein (e.g., GFP) integrated into the X chromosome. The remainder of the method is essentially identical to that described for FIG. 2.

Screening Assays

The single sex populations of non-human animals (e.g., Drosophila) produced by the methods of the present invention may, in a separate embodiment, be used in assays which, for example, screen for agents which modulate or modify a phenotype of the animals of the population, or which may be adapted to a microarray format for the analysis of gene transcription, or which assay for the expression of certain proteins by the population. That is, in one embodiment, the invention encompasses a method for identifying compounds which may be used to modulate or modify a phenotype, wherein the method comprises a first step of producing a single sex population of non-human animals according to the method of the invention.

In one embodiment, the pure single sex population of animals, preferably animal embryos and/or larvae, which result from the sorting method of the invention may be, for example, dissociated, and treated to extract DNA, RNA (which may be used to generate cDNA) which is then arrayed in a microarray which may be screened with nucleic acid probes to determine the expression of a gene of interest in the population. Methods for the preparation of DNA, RNA and cDNA samples from cell, tissue, organ, or whole animal (e.g., embryo and/or larvae) samples are well known in the art, and may be found, for example in Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987). Methods for producing microarrays of DNA, RNA, cDNA or tissue are known in the art, including substrates, gridding techniques, and methods for probing microarrays with DNA, RNA or cDNA nucleic acid probes (see, for example Fodor et al., U.S. Pat. No. 5,510,270; Lockhart et al., U.S. Pat. No. 5,556,752; Hybridization With Polynucleotide Probes, P. Tijssen, ed. Elsevier, N.Y., (1993)).

In a further embodiment, the single sex sorted population of non-human animals may be used according to the invention, in an assay to determine the expression of one or more proteins in the animals of the population, preferably, the embryos/larvae of the population. For example, arrays of antibodies may be used as a basis for screening populations of polypeptides derived from the sorted, sex-specific population. Examples of protein and antibody arrays are given in Proteomics: A Trends Guide, Elsevier Science Ltd., July 2000 which is incorporated by reference. Proteomics assay techniques are known in the art and may be readily adapted to the present invention (see, for example, Celis et al., 2000, FEBS Lett, 480(1):2-16; Lockhart and Winzeler, 2000, Nature 405(6788):827-836; Khan et al., 1999, 20(2):223-9). Proteomics applications involving mass spectrometry, peptide mass fingerprinting/protein identification, and protein quantification may also be performed using the sorted single-sex populations of the invention.

In a preferred embodiment, the sorted single-sex population of non-human animals (preferably Drosophila) are used to screen for agents which alter a phenotype of the animals of the population. Phenotypes which may be assayed according to the invention include observable and/or measurable physical, behavioral, or biochemical characteristics of a non-human animal useful in the invention (e.g., a fly). Phenotypic traits which may be measured according to the invention include, but are not limited to those described in WO 04/006854, published Jan. 22, 2004 (incorporated herein in its entirety). Accordingly, test agents may be assayed to determine whether they are capable of producing a change in phenotype in the population. An altered or changed phenotype includes a phenotype that has changed relative to the phenotype of a wild-type or control animal. Examples of altered or changed phenotypes include a behavioral phenotype, such as appetite, mating behavior, and/or life span, that has changed by a measurable amount, e.g. by at least 10%, 20%, 30%, 40%, or more preferably 50%, relative to the phenotype of a control animal (wherein a “control animal” refers to an animal which does not express a heterologous gene of interest, or which has not been exposed to a candidate agent); or a morphological phenotype that has changed in an observable way, e.g. different growth rate of the animal; or different shape, size, color, or location of an organ or appendage; or different distribution, and/or characteristic of a tissue, as compared to the shape, size, color, location of organs or appendages, or distribution or characteristic of a tissue observed in a control animal, wherein any statistically significant difference in the phenotype (when determined across a test population relative to a control population) is indicative of an altered phenotype. According to the present invention a phenotype characteristic associated with a neurodegenerative disease is said to be altered if the measurement of one or more of the characteristics is increased or decreased. That is, where a phenotype characteristic associated with a neurodegenerative disease is an abnormal phenotype (a phenotype which, when quantitiated by the methods of the invention is of a value which is different from the same phenotype measurement made in a control animal, wherein the difference is statistically significant (p≦0.05)) the abnormal phenotype is said to be altered when the phenotype is either increased (made more abnormal) or decreased (made less abnormal and closer to the phenotype measured from a control animal). According to the present invention, an abnormal phenotypic characteristic is considered to be “increased” where the particular characteristic becomes more severe (e.g., where the characteristic is premature death, the animal dies earlier; where the characteristic is the presence of nuclear inclusions, the animal has more nuclear inclusions per cell, or more cells with inclusions; where the characteristic is ataxia, the animal has more severe ataxia; etc.), that is, there is a statistically significant (p≦0.05) difference in the measurement of the characteristic at a first reference point and the measurement of the more severe characteristic at a second reference point. According to the present invention, an abnormal phenotypic characteristic is considered to be “decreased” where the particular characteristic becomes less severe (e.g., where the characteristic is premature death, the animal dies later; where the characteristic is the presence of nuclear inclusions, the animal has fewer nuclear inclusions per cell, or fewer cells with inclusions; where the characteristic is ataxia, the animal has less severe ataxia; etc.), that is, there is a statistically significant (p≦0.05) difference in the measurement of the characteristic at a first reference point and the measurement of the less severe characteristic a second reference point.

In a preferred embodiment, the present invention provides a method for screening for agents which may be active in a neurodegenerative disease; that is, may induce a difference in a neurodegeneraitve phenotype in an animal contacted with the agent relative to an animal which has not been contacted with the agent. The female and male generator populations described above may be adapted to express, for example via the Gal4/UAS system a heterologous neurodegenerative disease gene as defined herein. The result of including the Gal4/UAS system coupled to the male and female generator populations is that the final sorted single-sex population of non-human animals will express both the Gal4 driver and UAS/neurodegenerative disease gene targets, and will thus express the neurodegenerative disease gene. This population may be used to determine any difference in phenotype between the sorted population expressing the neurodegenerative disease gene and a control population (e.g., measuring a “difference phenotype”). This population may then be screened against test agents to determine whether the test agent is active in neurodegenerative disease, wherein the agent is deemed to be active in neurodegenerative disease if there is a change in the difference phenotype in the test population relative to a control population.

A change in an altered phenotype includes either complete or partial reversion of the phenotype observed (e.g., reversion of the altered phenotype in response to a test agent). Complete reversion is defined as the absence of the altered phenotype, or as 100% reversion of the phenotype to that phenotype observed in control flies. Partial reversion of an altered phenotype can be 5%, 10%, 20%, preferably 30%, more preferably 50%, and most preferably greater than 50% reversion to that phenotype observed in control flies. Example measurable parameters include, but are not limited to, size and shape of organs, such as the eye; distribution of tissues and organs; behavioral phenotypes (such as, appetite and mating); and locomotor ability, such as can be observed in a climbing assays as described below.

In a preferred embodiment, the non-human animals of the invention are Drosophila and altered locomoter phenotype is measured using a climbing assay. For example, in a climbing assay, locomotor ability can be assessed by placing flies in a vial, knocking them to the bottom of the vial, then counting the number of flies that climb past a given mark on the vial during a defined period of time. 100% locomotor activity of control flies is represented by the number of flies that climb past the given mark, while flies with an altered locomotor activity can have 80%, 70%,60%, 50%, preferably less than 50%, or more preferably less than 30% of the activity observed in a control fly population. Methods for measuring locomotive or climbing behavior of flies are described, for example, in U.S. 20040126319, published Jul. 1, 2004, and incorporated herein by reference.

In a further embodiment, the present invention may be used to screen for compounds which regulate or modulate other aspects of the physiology of members of a sorted population, such as cardiac function, hypoxia, or anoxia.

Test Agents

Agents that are useful in the screening assays described herein include biological or chemical agents that when administered to an animal have the potential to modify an altered phenotype, e.g. partial or complete reversion of the phenotype. Agents include any recombinant, modified or natural nucleic acid molecule; library of recombinant, modified or natural nucleic acid molecules; synthetic, modified or natural peptides; library of synthetic, modified or natural peptides; organic or inorganic compounds; or library of organic or inorganic compounds, including small molecules. Agents can also be linked to a common or unique tag, which can facilitate recovery of the therapeutic agent.

Example agent sources include, but are not limited to, random peptide libraries as well as combinatorial chemistry-derived molecular library made of D- and/or L-configuration amino acids; phosphopeptides (including, but not limited to, members of random or partially degenerate, directed phosphopeptide libraries; see, e.g., Songyang et al., Cell 72:767-778 (1993)); antibodies (including, but not limited to, polyclonal, monoclonal, humanized, anti-idiotypic, chimeric or single chain antibodies, and FAb, F(ab′)2 and FAb expression library fragments, and epitope-binding fragments thereof); and small organic or inorganic molecules. Examples of chemically synthesized libraries are described in Fodor et al., Science 251:767-773 (1991); Houghten et al., Nature 354:84-86 (1991); Lam et al., Nature 354:82-84 (1991); Medyuski, Bio/Technology 12:709-710 (1994); Gallop et al., J. Medicinal Chemistry 37(9):1233-1251 (1994); Ohlmeyer et al., Proc. Natl. Acad. Sci. USA 5 90: 10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614-1618 (1994); Salmon et al., Proc. Natl. Acad. Sci. USA 90:11708-11712 (1993); PCT Publication No. WO 93/20242; and Brenner and Lemer, Proc. Natl. Acad. Sci. USA 89:5381-5383 (1992). By way of examples of nonpeptide libraries, a benzodiazopine library (see e.g., Bunin et al., Proc. Natl. Acad. Sci. USA 91:4708-4712 (1994)) can be adapted for use. Other libraries of agents useful in the invention include peptide libraries (Simon et al., Proc. Natl. Acad. Sci. USA 89:9367-9371 (1992)), a combinatorial library (Ostresh et al. Proc. Natl. Acad. Sci. USA 91:11138-11142 (1994); Eichler & Houghten, Mol. Med. Today 1:174-180 (1995); Dolle, Mol. Divers. 2:223-236 (1997); and Lam, Anticancer Drug Des. 12:145-167 (1997).), phage display libraries wherein peptide libraries can be produced (Scott & Smith, Science 249:386-390 (1990); Devlin et al., Science, 249:404-406 (1990); Christian et al., J. Mol. Biol. 227:711-718 (1992); Lenska, J. Immunol. Meth. 152:149-157 (1992); Kay et al., Gene 128:59-65 (1993); and PCT Publication No. WO 94/18318 dated Aug. 18, 1994).

Agents that can be tested and identified by methods described herein can include, but are not limited to, compounds obtained from any commercial source, including Aldrich (Milwaukee, Wis. 53233), Sigma Chemical (St. Louis, Mo.), Fluka Chemie AG (Buchs, Switzerland) Fluka Chemical Corp. (Ronkonkoma, N.Y.), Eastman Chemical Company, Fine Chemicals (Kingsport, Tenn.), Boehringer Mannheim GmbH (Mannheim, Germany), Takasago (Rockleigh, N.J.), SST Corporation (Clifton, N.J.), Ferro (Zachary, La.), Riedel-deHaen AG (Seelze, Germany), PPG Industries Inc., Fine Chemicals (Pittsburgh, Pa.), Specs and BioSpecs B.V. (Rijswijk, The Netherlands), Chembridge Corporation (San Diego, Calif.), Contract Service Company (Dolgoprudoy, Moscow Region, Russia), Comgenex USA Inc. (Princeton, N.J.), Maybridge Chemicals Ltd. (Cornwall, United Kingdom), and Asinex (Moscow, Russia). Furthermore, any kind of natural products can be screened using the methods described herein, including microbial, fungal, plant or animal extracts.

Examples of methods for the synthesis of molecular libraries can be found in the art, for example in: DeWitt et al., Proc. Natl. Acad. Sci. USA 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and Gallop et al., 15 J. Med. Chem. 37:1233 (1994).

A library of agents can also be a library of nucleic acid molecules; DNA, RNA, or analogs thereof. For example, a cDNA library can be constructed from mRNA collected from a cell, tissue, organ or organism of interest, or genomic DNA can be treated to produce appropriately sized fragments using restriction endonucleases or methods that randomly fragment genomic DNA. A library containing RNA molecules can be constructed, for example, by collecting RNA from cells or by synthesizing the RNA molecules chemically. Diverse libraries of nucleic acid molecules can be made using solid phase synthesis, which facilitates the production of randomized regions in the molecules. If desired, the randomization can be biased to produce a library of nucleic acid molecules containing particular percentages of one or more nucleotides at a position in the molecule (U.S. Pat. No. 5,270,163).

A candidate agent can be administered by a variety of means. For example, where the non-human animal is an insect such as Drosophila, an agent can be administered by applying the candidate agent to the culture or rearing media. Alternatively, the candidate agent can be prepared in a 1% sucrose solution, and the solution fed to the animal for a specified time, such as 10 hours, 12 hours, 24 hours, 48 hours, or 72 hours.

In assays involving nematodes, the compounds to be tested are dissolved in DMSO or other organic solvent, mixed with a bacterial suspension at various test concentrations, preferably OP50 strain of bacteria (Brenner, Genetics (1974) 110:421-440), and supplied as food to the worms. The population of worms to be treated can be synchronized larvae (Sulston and Hodgkin, in The Nematode Caenorhabditis elegans (1988) (ed. Wood, W. B.) Cold Spring Harbor Laboratory) or adults or a mixed-stage population of animals.

Potential agents can be administered to the animal in a variety of ways, including orally (including addition to synthetic diet, application to plants or prey to be consumed by the test animal), topically (including immersion, painting, spraying, direct application of compound to animal, allowing animal to contact a treated surface), or by injection. Candidate agents are often hydrophobic molecules and must commonly be dissolved in organic solvents, which are allowed to evaporate in the case of methanol or acetone, or at low concentrations can be included to facilitate uptake (ethanol, dimethyl sulfoxide).

The candidate agent can be administered at any stage of animal development including fertilized eggs, embryonic, larval and adult stages. In one embodiment, the candidate agent is administered to an adult animal. In another embodiment, the candidate agent is administered during an embryonic or larval stage.

The agent can be administered in a single dose or multiple doses. Appropriate concentrations can be determined by one skilled in the art, and will depend upon the biological and chemical properties of the agent, the specific non-human animal to be assayed, as well as the method of administration. For example, concentrations of candidate agents can range from 0.0001 μM to 20 mM when delivered orally or through injection, 0.1 μM to 20 mM, 1 μM-10 mM, or 10 μM to 5 mM. In one embodiment, a test agent can be included in the rearing media at a concentration of between about 1 nM and 1 μM.

In a preferred embodiment, a high throughput screen of candidate agents is performed in which a large number of agents, at least 50 agents, 100 agents, or more than 100 agents are tested individually in parallel on a plurality of animal populations. An animal population contains at least 2, 10, 20, 50, 100, or more adult or juvenile animals.

In a preferred embodiment, the non-human animals of the invention are Drosophila and each test agent is brought into contact with the population of flies in a manner such that the active agent of the compound composition is capable of exerting activity on at least a substantial portion of, if not all of, the individual animals of the population. By substantial portion is meant at least 75%, usually at least 80% and in many embodiments can be as high as 90 or 95% or higher. Generally, the members of the population are contacted with each compound test agent in a manner such that the active agent of the composition is internalized by the flies. In some cases, internalization will be by ingestion, i.e. orally, such that that each compound composition will generally be contacted with the plurality of animals by incorporating the compound composition in a nutrient medium, e.g. water, yeast paste, aqueous solution of additional nutrient agents, etc., of the flies. For example, the candidate agent is generally orally administered to the fly by mixing the agent into the fly nutrient medium, such as a yeast paste, and placing the medium in the presence of the fly (either the larva or adult fly) such that the fly feeds on the medium. In some cases, flies of a population are contacted with a compound by exposing the population to the compound in the atmosphere, including vaporization or aerosol delivery of the compound, or spraying a liquid containing the compound onto the animals.

Upon administration of the candidate agent(s), the animal is then assayed for change in the phenotype, as described above, as compared to the phenotype displayed by a control animal that has not been administered a candidate agent (e.g., assaying for a change in the difference phenotype).

Mutation Analysis

In a further embodiment of the invention the sorting method shown in any of FIGS. 1-5 may be adapted to include a mutagenesis step. More specifically, where the male generator population comprises a UAS/heterologous gene target sequence integrated into the chromosome, once the population is induced to express the pro-apoptotic gene (or fluorescently sorted as shown in FIGS. 3-5), the resulting pure male population may be subjected to mutagenesis of the heterologous gene of interest, or may be subjected to random mutagenesis throughout the fly. The mutated population (population 4 in FIG. 1) is then crossed with the pure female Gal4 driver population (population 3 in FIG. 1), and a single-sex population of male or female animals (depending on which sex chromosome the fluorescent protein is integrated into; see above) comprising the mutation is then obtained The final sex-specific population comprising the mutation can then be assayed for phenotypic changes relative to populations which express the same heterologous gene not having a mutation. In this way, genetic modifiers of the heterologous gene of interest could be identified. Alternatively, the population comprising the mutation can be screened against one or more test agents to determine whether there is a change in phenotype relative to a population treated with the same test agent in the absence of the mutation. In this way, the cellular targets of the test agent could be identified.

Mutations can be introduced into non-human animals of the invention using methods which are known in the art. For example where the animal is a Drosophila, the animal can be mutagenized using chemicals, radiation or insertions (e.g. transposons, such as P-element mutagenesis), appropriate crosses performed, and the progeny screened for phenotypic differences in, for example, geotatic behavior compared with normal controls. The gene can then be identified by a variety of methods including, for example, linkage analysis or rescue of the gene targeted by the inserted element. Methods of mutating and identifying genes are described, for example, in Ashburner, In Drosophila melanogaster: A Laboratory Manual (1989) Greenspan, Cold Spring Harbor Laboratory Press, Fly Pushing: The Theory and Practice of Drosophila Genetics, Cold Spring Harbor Laboratory Press (1997), in R. K. Herman, Genetics: The Nematode Caenorhabditis elegans (1988) (ed. Wood, W. B.) Cold Spring Harbor Laboratory, or in Zebrafish: A Practical Approach (2002) (eds.: Nusslein-Volhard & Dahm), Oxford University Press), which are herein incorporated by reference.

Pharmaceutical Formulations

The invention further provides for (i) the use of agents identified by the above-described screening assays for treatment of disease in mammal, e.g., humans, domestic animals, livestock, pets, farm animals, or wildlife populations, (ii) pharmaceutical compositions comprising an agent identified by the above-described screening assay and (iii) methods for treating a mammal, e.g., humans, domestic animals, livestock, pets, farm animals, or wildlife populations that have a disease by administering an agent identified by the above-described screening assays. In one embodiment, the invention provides a method of preparing a medicament for use in treatment of a disease in mammals by (a) providing a population of flies with characteristics of a mammalian disease (e.g., flies which express a neurodegenerative disease gene operably linked to a UAS element) (b) using a method described herein to identify an agent expected to reduce the disease phenotype and (c) formulating the agent for administration to a mammal. In some cases, the phenotype of the population of flies in step (a) may be characteristic of a mammalian neurodegenerative disease. The population of flies in step (a) may be transgenic flies and, in some cases, the expression of the transgene may result in neurodegeneration or a phenotype of a neurodegenerative disease. Genes and transgenes associated with mammalian neurodegenerative diseases and flies containing such transgenes are described herein.

In one aspect, a method of preparing a medicament for use in treating a disease is provided, comprising formulating the agent for administration to a mammal, e.g., primate. For example, suitable formulations may be sterile and/or substantially isotonic and/or in full compliance with all Good Manufacturing Practice (GMP) regulations of the U.S. Food and Drug Administration and/or in a unit dosage form. See, Remington's Pharmaceutical Sciences (17th ed.) Mack Publishing Co., Easton, Pa.; Avis et al (eds.) (1993).

The invention will now be further described by way of Examples, which are meant to serve to assist one of ordinary skill in the art in carrying out the invention and are not intended in any way to limit the scope of the invention.

### EXAMPLES

### Example 1

### Generation of Drosophila Stocks

Female generator population

A female generator strain was produced to permit the culling of male flies, and to yield a pure population of female flies. Male flies of the female generator strain comprise a hid element under the control of a heat shock promoter integrated into the Y chromosome and optionally, a Gal4 driver sequence under the control of a tissue specific, or inducible promoter (i.e., elav) integrated into the X chromosome. To establish the Phs-hidY:elavGAL4 c155 stock, elavGAL4 c155 virgins were crossed to P[hs-hid,w⁺]Y/y,w males and subsequent male F1 progeny of genotype P[hs-hid,w⁺]Y/elavGAL4 c155 were backcrossed to elavGAL4 c155 virgins. The elavGAL4 c155 stock was obtained from Bloomington Stock Center, while the P[hs-hid,w⁺]Y/yw stock was obtained from Ruth Lehmann's lab (Howard Hughes Medical Institute). All crosses were carried out at 25° C.

Male Generator Population

A male generator strain was produced to permit the culling of female Drosophila to yield a pure population of male flies. A transposition screen was carried out to mobilize the P[w+,hs-hid] insert from a 3^{rd} chromosome balancer to an attached-X chromosome. In brief, C(1)DX,y,w,f/Y +/TM3,Sb,P[w+,hs-hid] virgins were crossed to y,w; Ki [Δ2-3] males, and then F1 C(1)DXy,w,f/Y; Ki [Δ2-3] /TM3,Sb,P[w+,hs-hid] female progeny were backcrossed to yw males to eliminate the transposase. 500 F2 single pair matings of genotype C(1)DXy,w,f/Y; +/TM3,Sb,P[w+,hs-hid] x y,w were set-up to identify lines, in which the w+ marker segregated with females, which is indicative of a P[w+,hs-hid] transposition event to the attached-X chromosome. Positive C(1)DXy,w,f,P[w+,hs-hid] strains, were subsequently crossed to y,w,P[actin-GFP,w+] /Y or y,w,P[actin-GFP,w+],fs(1)K10 males to establish the final male generator strain. All stocks were obtained from the Bloomington stock center (Indiana University). Crosses were carried out at 23° C.

Integration of Recessive Female Sterile Mutation onto the X

Attendant to the use of the attached-X genotype for the male generator population is the occurrence of non-dysjunction events in males, leading to the production of females which are fertile and not attached-X. The occurrence of such non-dysjunction events is rare, and takes place with a frequency of only 1/2000 to 1/5000. Regardless however, the rare occurrence of such a fertile female in the male generator population would impair the ability to produce a pure male population as these females would not carry the pro-apoptotic gene. Thus, in one embodiment, a female sterile mutation is integrated into the X-chromosome, such that in any non-dysjunction event, the X chromosome carrying the recessive female sterile mutation will be segregated to the female animals ensuring that females that arise from non-dysjunction events are infertile and cannot contaminate the population. To produce the GFP-tagged, recessive female sterile containing, X chromosome used in the male generator population, P[actin-GFP, w⁺] was mobilized from FM7i P[w⁺,actin-GFP] to a y,w, chromosome. Subsequently, y,w,P[actin-GFP,w⁺], was recombined with y,w,fs1(K10),P [ry+,neoFRT] and a y,w,P[w+,actin-GFP], fs(1)K10/y,w,f,C(1)DX stock was established. The y,w,P[w⁺,actin-GFP],fs(1)K10 chromosome was then crossed into the male generator background.

### Example 2

### Sex-Specific Sorting

Overview of Sex Sorting Experiment (See, e.g., FIG. 1)

GAL4 driver (female generator population) and effector (male generator population) lines are amplified to 10's of trays of each. GAL4 driver virgins and UAS effector males are isolated through a heat-shock event and then crossed together in a population cage to enable breading and to facilitate egg collection. Eggs are harvested and COPAS sorted into 16 mm assay vials. Eggs develop into adult flies, which are then assayed. Experimental controls that are included to monitor activity of the GAL4/UAS system include: GAL4/yw negative control, GAL4:UAS-GFP positive control.

Set-up Parental Strains and Heat-Shock. (Day 1-7)

Day 1:

8 trays (25 bottles per) of Elav-GAL4 c155:Y,P[hs-hid,w⁺] female generator stock (see Example 1) are transferred to fresh bottles. 4 trays of y,wf,C(1)DXP[hs-hid,w⁺]:actin-GFP:fs(1: UAS-Effector male generator stock (see Example 2) are transferred to fresh bottles and reared at 25° C.

Day 3:

Parents are transferred to fresh bottles to perpetuate stock for future experiments.

Day 6-7:

Male and Female generator bottles (now containing larvae) are heat-shocked on days 6 and 7 for 2 hours/day in a circulating 37° C. water bath. Bottles are submerged to the ‘buzz-plug’ to ensure maximal larval exposure.

Day 7-11:

Pupae mature to adults at 25° C.

Set-up F1 Cross and COPAS sort (Day 12-Day 26)

Combine 8 trays of Elav-GAL4 c155 virgin females with 4 trays of actin-GFP:fs(1)K10:UAS-Effector males in a population cage. Allow to mate for 2-3 days, before collecting embryos for COPAS sorting. Change grape plates (coated in yeast paste) twice daily, to promote egg laying. Sort 16-22 hr embryos with the COPAS (i.e. collect from 1 pm-7 pm the day before the COPAS run, age the embryos overnight, then process them by 10 am the following day). To prepare embryos for COPAS sorting, dechorionate embryos in 50% bleach for 5 min. and pass them through a sequential series of sieves (850 um, 425 um, 125 um) to eliminate yeast particulate matter and larvae. Rinse embryos in washing solution (0.7%/NaCl, 0.03% Triton X-100), and resuspended them in 50 mLs of Embryo Sample Solution (P/N 335-5075-000 Union Biometrica), in order to run them through the COPAS. COPAS sorting is performed according to the manufacturer's instructions and guidelines (Union Biometrica, Somerville, Mass.). 12 embryos/tube are sorted into 96 tube arrays of proprietary EnVivo16 mm vials containing 1.5 mL of Harvard media. Vials are subsequently capped and transferred to 25° C. to enable embryo maturation. Sorted embryos may be matured and used in numerous phenotype assays including, for example, a negative geotaxis assay to screen for locomotive defects.

Negative Geotaxis Assay (Day 27-Day 41)

Mature flies are transferred to assay vials containing a defined screening media and test compounds. Flies are flipped daily to fresh media/compound, and are assayed daily for two weeks. In a negative geotaxis assay (climbing assay), locomotor ability can be assessed by placing flies in a vial, knocking them to the bottom of the vial, then counting the number of flies that climb past a given mark on the vial during a defined period of time. 100% locomotor activity of control flies is represented by the number of flies that climb past the given mark, while flies with an altered locomotor activity can have 80%, 70%, 60%, 50%, preferably less than 50%, or more preferably less than 30% of the activity observed in a control fly population.

All patents, patent applications, and published references cited herein are hereby incorporated by reference in their entirety. While this invention has been particularly shown and described with references to preferred embodiments thereof, it will be understood by those of ordinary skill in the art that various changes in form and details may be made herein without departing from the scope of the invention encompassed by the following claims.

1. A population of male and female non-human animals wherein said male animals comprise a pro-apoptotic gene operably linked to a regulatable promoter integrated into the Y chromosome, wherein said regulatable promoter is not a heat-shock promoter.

2. The population of claim 1, wherein said pro-apoptotic gene is selected from the group consisting of head involution defective, reaper, grim, hid-ala, ICE, and ced-3.

3. The population of claim 1, wherein said animals are selected from the group consisting of Drosophila, silkworm, C. elegans, zebrafish, zooplankton, medakafly, mosquito, and xenopus.

4. The population of claim 1, wherein said animals are Drosophila.

5. A population of male and female non-human animals wherein said male animals comprise a pro-apoptotic gene operably linked to a regulatable promoter integrated into the Y chromosome and further comprises, integrated into the genome of said male and female non-human animals a sequence encoding Gal4 operably linked to a neuronal or glial-specific promoter.

6. The population of claim 5, wherein said pro-apoptotic gene is selected from the group consisting of head involution defective, reaper, grim, hid-ala, ICE, and ced-3.

7. The population of claim 5, wherein said regulatable promoter is selected from the group consisting of heat shock promoter, Gal40, Gal80, Tet, and RU486.

8. The population of claim 5, wherein said animals are selected from the group consisting of Drosophila, silkworm, C. elegans, zebrafish, zooplankton, medakafly, mosquito, and xenopus.

9. The population of claim 5, wherein said animals are Drosophila.

10. A population of insects comprising male and female insects wherein said male insects comprise a pro-apoptotic gene operably linked to a regulatable promoter integrated into the Y chromosome; and, wherein said population further comprises, integrated into the genome of said male and female insects a nucleic acid sequence encoding Gal4 operably linked to a neuronal or glial-specific promoter.

11. The population of claim 10, wherein said pro-apoptotic gene is selected from the group consisting of head involution defective, reaper, grim, hid-ala, ICE, and ced-3.

12. The population of claim 10, wherein said regulatable promoter is selected from the group consisting of heat shock promoter, Gal40, Gal80, Tet, and RU486.

13. The population of claim 10, wherein said insects are selected from the group consisting of Drosophila, silkworm, and mosquito.

14. The population of claim 10, wherein said insects are Drosophila.

15. A population of Drosophila comprising male and female Drosophila, wherein said male Drosophila comprise a pro-apoptotic gene operably linked to a regulatable promoter integrated into the Y chromosome; and said population further comprises, integrated into the genome of said male and female Drosophila, a nucleic acid sequence encoding Gal4 operably linked to a neuronal or glial-specific promoter.

16. The population of claim 15, wherein said pro-apoptotic gene is selected from the group consisting of head involution defective, reaper, grim, hid-ala, ICE, and ced-3.

17. The population of claim 15, wherein said regulatable promoter is selected from the group consisting of heat shock promoter, Gal40, Gal80, Tet, and RU486.

18. A population of male and female non-human animals wherein said female non-human animals comprise an attached-X chromosome, and wherein a pro-apoptotic gene is integrated into said attached-X chromosome.

19. The population of claim 18, wherein said pro-apoptotic gene is selected from the group consisting of head involution defective, reaper, grim, hid-ala, ICE, and ced-3.

20. The population of claim 18, wherein said pro-apoptotic gene is operably linked to a regulatable promoter.

21. The population of claim 21, wherein said regulatable promoter is selected from the group consisting of heat shock promoter, Gal40, Gal80, Tet, and RU486.

22. The population of claim 18, wherein said male non-human animals of said population comprise a sequence encoding a fluorescent protein integrated into the X chromosome.

23. The population of claim 18, wherein said animals further comprise, integrated into the X chromosome, a female sterile mutation.

24. The population of claim 23, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

25. The population of claim 18, wherein said non-human animals are selected from the group consisting of Drosophila, silkworm, C. elegans, zebrafish, zooplankton, medaka, mosquito, and xenopus.

26. The population of claim 18, wherein said male and female non-human animals further comprises an upstream activator sequence operably linked to a neurodegenerative disease gene.

27. A population of male and female non-human animals wherein said female animals comprise an attached-X chromosome, and wherein a pro-apoptotic gene is integrated into said attached-X chromosome, and wherein said male and female non-human animals further comprise an upstream activator sequence operably linked to a heterologous gene of interest.

28. The population of claim 27, wherein said pro-apoptotic gene is selected from the group consisting of head involution defective, reaper, grim, hid-ala, ICE, and ced-3.

29. The population of claim 27, wherein said pro-apoptotic gene is operably linked to a regulatable promoter.

30. The population of claim 29, wherein said regulated promoter is selected from the group consisting of heat shock promoter, Gal40, Gal80, Tet, and RU486.

31. The population of claim 27, wherein said male animals of said population comprise a sequence encoding a fluorescent protein integrated into the X chromosome.

32. The population of claim 27, wherein said animals further comprise, integrated into the X chromosome, a female sterile mutation.

33. The population of claim 32, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

34. The population of claim 27, wherein said animals are selected from the group consisting of Drosophila, silkworm, C. elegans, zebrafish, zooplankton, medaka, mosquito, and xenopus.

35. A population of male and female non-human animals, wherein said female animals comprise an attached-X chromosome, and wherein a pro-apoptotic gene is integrated into said attached-X chromosome, and wherein said male and female non-human animals further comprise an upstream activator sequence operably linked to a heterologous gene of interest, and said male and female animals further comprises a sequence encoding a fluorescent protein integrated into a sex chromosome.

36. The population of claim 35, wherein said pro-apoptotic gene is selected from the group consisting of head involution defective, reaper, grim, hid-ala, ICE, and ced-3.

37. The population of claim 35, wherein said pro-apoptotic gene is operably linked to a regulatable promoter.

38. The population of claim 37, wherein said regulatable promoter is selected from the group consisting of heat shock promoter, Gal40, Gal80, Tet, and RU486.

39. The population of claim 35, wherein said animals further comprise, integrated into the X chromosome, a female sterile mutation.

40. The population of claim 39, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

41. The population of claim 35, wherein said animals are selected from the group consisting of Drosophila, silkworm, C. elegans, zebrafish, zooplankton, medaka, mosquito, and xenopus.

42. A population of male and female non-human animals, wherein said female animals comprises an attached-X chromosome, and wherein a pro-apoptotic gene is integrated into said attached-X chromosome, and wherein said male and female animals further comprise an upstream activator sequence operably linked to a heterologous gene of interest, and wherein said male and female animals further comprise a sequence encoding a fluorescent protein integrated into a sex chromosome, and wherein said population of non-human animals further comprises, integrated in the X chromosome, a female sterile mutation.

43. The population of claim 42, wherein said pro-apoptotic gene is selected from the group consisting of head involution defective, reaper, grim, hid-ala, ICE, and ced-3.

44. The population of claim 42, wherein said pro-apoptotic gene is operably linked to a regulatable promoter.

45. The population of claim 44, wherein said regulatable promoter is selected from the group consisting of heat shock promoter, Gal40, Gal80, Tet, and RU486.

46. The population of claim 42, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

47. The population of claim 42, wherein said animals are selected from the group consisting of Drosophila, silkworm, C. elegans, zebrafish, zooplankton, medaka, mosquito, and xenopus.

48. A population of insects comprising male and female insects wherein said female insects comprises an attached-X chromosome, and wherein a pro-apoptotic gene is integrated into said attached-X chromosome.

49. The population of claim 48, wherein said male insects comprise a sequence encoding a fluorescent protein integrated into the X chromosome.

50. The population of claim 48, wherein said pro-apoptotic gene is selected from the group consisting of head involution defective, reaper, grim, hid-ala, ICE, and ced-3.

51. The population of claim 48, wherein said pro-apoptotic gene is operably linked to a regulatable promoter.

52. The population of claim 51, wherein said regulatable promoter is selected from the group consisting of heat shock promoter, Gal40, Gal80, Tet, and Ru486.

53. The population of claim 48, wherein said insects further comprise, integrated into the X chromosome, a female sterile mutation.

54. The population of claim 53, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

55. The population of claim 48, wherein said insects are selected from the group consisting of Drosophila, silkworm, and mosquito.

56. The population of claim 48; wherein said male and female insects further comprises an upstream activator sequence operably linked to a neurodegenerative disease gene.

57. A population of Drosophila comprising male and female Drosophila wherein said female Drosophila comprises an attached-X chromosome, and wherein a pro-apoptotic gene is integrated into said attached-X chromosome.

58. The population of claim 57, wherein said pro-apoptotic gene is selected from the group consisting of head involution defective, reaper, grim, hid-ala, ICE, and ced-3.

59. The population of claim 57, wherein said pro-apoptotic gene is operably linked to a regulatable promoter.

60. The population of claim 59, wherein said regulatable promoter is selected from the group consisting of heat shock promoter, Gal40, Gal80, Tet, and RU486.

61. The population of claim 57, wherein said Drosophila further comprise, integrated into the X chromosome, a female sterile mutation.

62. The population of claim 61, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

63. The population of claim 57, wherein said male Drosophila comprise a sequence encoding a fluorescent protein integrated into the X chromosome

64. The population of claim 57, wherein said male and female insects further comprises an upstream activator sequence operably linked to a neurodegenerative disease gene.

65. A method for producing a population of female insects, comprising:
(a) preparing a first population of insects comprising male and female insects wherein said male insects comprise a pro-apoptotic gene operably linked to a regulatable promoter integrated into the Y chromosome;
(b) preparing a second population of insects comprising male and female insects wherein said female insects comprise an attached-X chromosome, and wherein a pro-apoptotic gene operably linked to a regulatable promoter is integrated into said attached-X chromosome, and wherein said male insects of said second population comprise a nucleic acid sequence encoding a fluorescent protein integrated into the X chromosome;
(c) inducing said regulatable promoter in said first and second populations of insects such that a third population of insects comprising the female insects of said first population is produced, and a fourth population of insects comprising the male insects of said second population is produced;
(d) crossing said third and fourth population of insects to produce a fifth population of insects comprising male and female insects; and
(e) selecting female insects from said fifth population of insects.

66. The method of claim 65, wherein said regulatable promoter is selected from the group consisting of heat shock promoter, Gal40, Gal80, Tet, and RU486.

67. A method for producing a population of female insects comprising a heterologous gene of interest, comprising:
(a) preparing a first population of insects comprising male and female insects wherein said male insects comprise a pro-apoptotic gene operably linked to a regulatable promoter integrated into the Y chromosome;
(b) preparing a second population of insects comprising male and female insects wherein said female insects comprise an attached-X chromosome, and wherein a pro-apoptotic gene operably linked to a regulatable promoter is integrated into said attached-X chromosome, and wherein said male insects of said second population comprise a sequence encoding a fluorescent protein integrated into the X chromosome;
(c) inducing said regulatable promoter in said first and second populations of insects such that a third population of insects comprising the female insects of said first population is produced, and a fourth population of insects comprising the male insects of said second population is produced;
(d) crossing said third and fourth population of insects to produce a fifth population of insects comprising male and female insects; and
(e) selecting female insects comprising said heterologous gene of interest from said fifth population of insects.

68. The method of claim 67, wherein said regulatable promoter is selected from the group consisting of heat shock promoter, Gal40, Gal80, Tet, and RU486.

69. The method of claim 67, wherein said male and female insects of said first population further comprises a sequence encoding yeast Gal4.

70. The method of claim 67, wherein said male and female insects of said second population further comprises an upstream activator sequence operably linked to said heterologous gene of interest

71. The method of claim 67, wherein said insects of said fifth population are insect embryos.

72. The method of claim 67, wherein said female insects of said fifth population express said fluorescent protein.

73. The method of claim 67, wherein step (e) comprises selecting female insects which express said fluorescent protein.

74. The method of claim 67, wherein said step (e) comprises selecting female insects using flow cytometry.

75. The method of claim 74, wherein said flow cytometry is performed using a complex object parametric analyzer and sorter

76. The method of claim 67, wherein said insects are selected from the group consisting of Drosophila, silkworm, and mosquito.

77. The method of claim 67, wherein said pro-apoptotic gene is selected from the group consisting of head involution defective, reaper, grim, hid-ala, ICE, and ced-3.

78. The method of claim 67, wherein said sequence encoding a fluorescent protein encodes a green fluorescent protein.

79. The method of claim 67, wherein said heterologous gene of interest is a neurodegenerative disease gene.

80. The method of claim 67, wherein said insects of said second population further comprise, integrated into the X chromosome, a female sterile mutation.

81. The method of claim 80, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

82. The method of claim 67, wherein said fifth population of insects is placed in contact with rearing media comprising one or more test compounds.

83. A method for producing a population of female Drosophila comprising a heterologous gene of interest, comprising:
(a) preparing a first population of Drosophila comprising male and female Drosophila wherein said male Drosophila comprise a pro-apoptotic gene operably linked to a regulatable promoter integrated into the Y chromosome;
(b) preparing a second population of Drosophila comprising male and female Drosophila wherein said female Drosophila comprise an attached-X chromosome, and wherein a pro-apoptotic gene operably linked to a regulatable promoter is integrated into said attached-X chromosome, and wherein said male Drosophila of said second population comprise a sequence encoding a fluorescent protein integrated into the X chromosome;
(c) inducing said regulatable promoter in said first and second populations of Drosophila such that a third population of Drosophila comprising the female Drosophila of said first population is produced, and a fourth population of Drosophila comprising the male Drosophila of said second population is produced;
(d) crossing said third and fourth population of Drosophila to produce a fifth population of Drosophila comprising male and female Drosophila; and
(e) selecting female Drosophila comprising said heterologous gene of interest from said fifth population of Drosophila.

84. The method of claim 83, wherein said regulatable promoter is selected from the group consisting of heat shock promoter, Gal40, Gal80, Tet, and RU486.

85. The method of claim 83, wherein said male and female Drosophila of said first population further comprises a sequence encoding yeast Gal4

86. The method of claim 83, wherein said male and female Drosophila of said second population further comprises an upstream activator sequence operably linked to said heterologous gene of interest

87. The method of claim 83, wherein said insects of said fifth population are Drosophila embryos.

88. The method of claim 83, wherein said female Drosophila of said fifth population express said fluorescent protein.

89. The method of claim 83, wherein step (e) comprises selecting female Drosophila which express said fluorescent protein.

90. The method of claim 83 wherein said step (e) comprises selecting Drosophila insects using flow cytometry.

91. The method of claim 90 wherein said flow cytometry is performed using a complex object parametric analyzer and sorter

92. The method of claim 83, wherein said pro-apoptotic gene is selected from the group consisting of head involution defective, reaper, grim, hid-ala, ICE, and ced-3.

93. The method of claim 83, wherein said sequence encoding a fluorescent protein encodes a green fluorescent protein.

94. The method of claim 83, wherein said heterologous gene of interest is a neurodegenerative disease gene.

95. The method of claim 83, wherein said Drosophila of said second population further comprise, integrated into the X chromosome, a female sterile mutation.

96. The method of claim 94, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

97. The method of claim 83, wherein said fifth population of Drosophila is placed in contact with rearing media comprising one or more test compounds.

98. A method for producing a population of female insects comprising a heterologous gene of interest, comprising:
(a) preparing a first population of insects comprising male and female insects wherein said male insects comprise a pro-apoptotic gene operably linked to a regulatable promoter integrated into the Y chromosome, and wherein said male and female insects further comprises a sequence encoding yeast Gal4;
(b) preparing a second population of insects comprising male and female insects wherein said female insects comprise an attached-X chromosome, and wherein a pro-apoptotic gene operably linked to a regulatable promoter is integrated into said attached-X chromosome, and wherein said male and female insects further comprises an upstream activator sequence operably linked to a heterologous gene of interest, and wherein said male insects of said second population comprise a sequence encoding a fluorescent protein integrated into the X chromosome;
(c) inducing said regulatable promoter of said first and second populations such that a third population of insects comprising the female insects of said first population is produced, and a fourth population of insects comprising the male insects of said second population is produced;
(d) crossing said third and fourth population of insects to produce a fifth population of insects comprising male and female insects; and
(e) selecting female insects comprising said heterologous gene of interest from said fifth population of insects.

99. The method of claim 98, wherein said regulatable promoter is selected from the group consisting of heat shock promoter, Gal40, Gal80, Tet, and RU486.

100. The method of claim 98, wherein said insects of said fifth population are insect embryos.

101. The method of claim 98, wherein said female insects of said fifth population express said fluorescent protein.

102. The method of claim 98, wherein step (e) comprises selecting female insects which express said fluorescent protein.

103. The method of claim 98 wherein said step (e) comprises selecting female insects using flow cytometry.

104. The method of claim 103, wherein said flow cytometry is performed using a complex object parametric analyzer and sorter

105. The method of claim 98, wherein said insects are selected from the group consisting of Drosophila, silkworm, and mosquito.

106. The method of claim 98, wherein said pro-apoptotic gene is selected from the group consisting of head involution defective, reaper, grim, hid-ala, ICE, and ced-3.

107. The method of claim 98, wherein said sequence encoding a fluorescent protein encodes a green fluorescent protein.

108. The method of claim 98, wherein said heterologous gene of interest is a neurodegenerative disease gene.

109. The method of claim 98, wherein said insects of said second population further comprise, integrated into the X chromosome, a female sterile mutation.

110. The method of claim 109, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

111. The method of claim 98, wherein said fifth population of insects is placed in contact with rearing media comprising one or more test compounds.

112. A method for producing a population of female Drosophila comprising a heterologous gene of interest, comprising:
(a) preparing a first population of Drosophila comprising male and female Drosophila wherein said male Drosophila comprise a pro-apoptotic gene operably linked to a regulatable promoter integrated into the Y chromosome, and wherein said male and female Drosophila further comprises a sequence encoding yeast Gal4;
(b) preparing a second population of Drosophila comprising male and female Drosophila wherein said female Drosophila comprise an attached-X chromosome, and wherein a pro-apoptotic gene operably linked to a regulatable promoter is integrated into said attached-X chromosome, and wherein said male and female Drosophila further comprise an upstream activator sequence operably linked to a heterologous gene of interest, and wherein said male Drosophila of said second population comprise a sequence encoding a fluorescent protein integrated into the X chromosome;
(c) inducing said regulatable promoter in said first and second populations of Drosophila such that a third population of Drosophila comprising the female Drosophila of said first population is produced, and a fourth population of Drosophila comprising the male Drosophila of said second population is produced;
(d) crossing said third and fourth population of Drosophila to produce a fifth population of Drosophila comprising male and female Drosophila; and
(e) selecting female Drosophila comprising said heterologous gene of interest from said fifth population of Drosophila.

113. The method of claim 112, wherein said regulatable promoter is selected from the group consisting of heat shock promoter, Gal40, Gal80, Tet, and RU486.

114. The method of claim 112, wherein said insects of said fifth population are Drosophila embryos.

115. The method of claim 112, wherein said female Drosophila of said fifth population express said fluorescent protein.

116. The method of claim 112, wherein step (e) comprises selecting female Drosophila which express said fluorescent protein.

117. The method of claim 116 wherein said step (e) comprises selecting Drosophila insects using flow cytometry.

118. The method of claim 114, wherein said flow cytometry is performed using a complex object parametric analyzer and sorter

119. The method of claim 112, wherein said pro-apoptotic gene is selected from the group consisting of head involution defective, reaper, grim, hid-ala, ICE, and ced-3.

120. The method of claim 112, wherein said sequence encoding a fluorescent protein encodes a green fluorescent protein.

121. The method of claim 112, wherein said heterologous gene of interest is a neurodegenerative disease gene.

122. The method of claim 112, wherein said Drosophila of said second population further comprise, integrated into the X chromosome, a female sterile mutation.

123. The method of claim 122 wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

124. The method of claim 112, wherein said fifth population of Drosophila is placed in contact with rearing media comprising one or more test compounds.

125. A method for producing a population of male insects, comprising:
(a) preparing a first population of insects comprising male and female insects wherein said male insects comprise a pro-apoptotic gene operably linked to a regulatable promoter integrated into the Y chromosome;
(b) preparing a second population of insects comprising male and female insects wherein said male insects comprise a sequence encoding a fluorescent protein integrated into the Y chromosome;
(c) inducing said regulatable promoter in said first population such that a third population of insects comprising the female insects of said first population is produced;
(d) selecting from said second population, male insects which express said fluorescent protein such that a fourth population of insects comprising the male insects of said second population is produced;
(e) crossing said third and fourth population of insects to produce a fifth population of insects comprising male and female insects; and
(f) selecting male insects from said fifth population of insects.

126. The method of claim 125, wherein said regulatable promoter is selected from the group consisting of heat shock promoter, Gal40, Gal80, Tet, and RU486.

127. A method for producing a population of male insects comprising a heterologous gene of interest, comprising:
(a) preparing a first population of insects comprising male and female insects wherein said male insects comprise a pro-apoptotic gene operably linked to a regulatable promoter integrated into the Y chromosome, wherein said male and female insects further comprises a sequence encoding yeast Gal4;
(b) preparing a second population of insects comprising male and female insects wherein said male insects comprise a sequence encoding a fluorescent protein integrated into the Y chromosome, and wherein said male and female insects further comprise an upstream activator sequence operably linked to a heterologous gene of interest;
(c) inducing said regulatable promoter in said first population such that a third population of insects comprising the female insects of said first population is produced;
(d) selecting from said second population, male insects which express said fluorescent protein such that a fourth population of insects comprising the male insects of said second population is produced;
(e) crossing said third and fourth population of insects to produce a fifth population of insects comprising male and female insects; and
(f) selecting male insects comprising said heterologous gene of interest from said fifth population of insects.

128. The method of claim 127, wherein said regulatable promoter is selected from the group consisting of heat shock promoter, Gal40, Gal80, Tet, and RU486.

129. The method of claim 127, wherein said insects of said fifth population are insect embryos.

130. The method of claim 127, wherein said male insects of said fifth population express said fluorescent protein.

131. The method of claim 127, wherein step (f) comprises selecting male insects which express said fluorescent protein.

132. The method of claim 131 wherein said step (f) comprises selecting male insects using flow cytometry.

133. The method of claim 132, wherein said flow cytometry is performed using a complex object parametric analyzer and sorter

134. The method of claim 127, wherein said insects are selected from the group consisting of Drosophila, silkworm, and mosquito.

135. The method of claim 127, wherein said pro-apoptotic gene is selected from the group consisting of head involution defective, reaper, grim, hid-ala, ICE, and ced-3.

136. The method of claim 127, wherein said sequence encoding a fluorescent protein encodes a green fluorescent protein.

137. The method of claim 127, wherein said heterologous gene of interest is a neurodegenerative disease gene.

138. A method for producing a humanized population of female insects, comprising:
(a) preparing a first population of insects comprising male and female insects wherein said male insects comprise a pro-apoptotic gene operably linked to a regulatable promoter integrated into the Y chromosome, and wherein said male and female insects further comprises a sequence encoding yeast Gal4;
(b) preparing a second population of insects comprising male and female insects wherein said female insects comprise an attached-X chromosome, and wherein a pro-apoptotic gene operably linked to a regulatable promoter is integrated into said attached-X chromosome, and wherein said male and female insects further comprise an upstream activator sequence operably linked to a human gene of interest, and wherein said male insects of said second population comprise a sequence encoding a fluorescent protein integrated into the X chromosome;
(c) inducing said regulatable promoter in said first and second populations such that a third population of insects comprising the female insects of said first population is produced, and a fourth population of insects comprising the male insects of said second population is produced;
(d) crossing said third and fourth population of insects to produce a fifth population of insects comprising male and female insects; and
(e) selecting humanized female insects comprising said human gene of interest from said fifth population of insects.

139. The method of claim 138, wherein said regulatable promoter is selected from the group consisting of heat shock promoter, Gal40, Gal80, Tet, and RU486.

140. The method of claim 138, wherein said insects of said fifth population are insect embryos.

141. The method of claim 138, wherein said female insects of said fifth population express said fluorescent protein.

142. The method of claim 138, wherein step (e) comprises selecting female insects which express said fluorescent protein.

143. The method of claim 142 wherein said step (e) comprises selecting female insects using flow cytometry.

144. The method of claim 143, wherein said flow cytometry is performed using a complex object parametric analyzer and sorter

145. The method of claim 138, wherein said insects are selected from the group consisting of Drosophila, silkworm, and mosquito.

146. The method of claim 138, wherein said pro-apoptotic gene is selected from the group consisting of head involution defective, reaper, grim, hid-ala, ICE, and ced-3.

147. The method of claim 138, wherein said sequence encoding a fluorescent protein encodes a green fluorescent protein.

148. The method of claim 138, wherein said human gene of interest is a neurodegenerative disease gene.

149. The method of claim 138, wherein said insects of said second population further comprise, integrated into the X chromosome, a female sterile mutation.

150. The method of claim 149, wherein said female sterile mutation is selected from the group consisting of fs(1)K10, JA127, JC105, EC205, EA130, RC63, VA296 DF942, DC776, HA90, L271, VA172, JC155, DC798, HF330, HF311, ED226, EF462, D62, D72, EA75, gt^{xll}, and fs(1)pcx.

151. A method for producing a humanized population of male insects, comprising:
(a) preparing a first population of insects comprising male and female insects wherein said male insects comprise a pro-apoptotic gene operably linked to a regulatable promoter integrated into the Y chromosome, and wherein said male and female insects further comprises a sequence encoding yeast Gal4;
(b) preparing a second population of insects comprising male and female insects wherein said male insects comprise a sequence encoding a fluorescent protein integrated into the Y chromosome, and wherein said male and female insects further comprises an upstream activator sequence operably linked to a human gene of interest;
(c) inducing said regulatable promoter in said first population such that a third population of insects comprising the female insects of said first population is produced;
(d) selecting from said second population, male insects which express said fluorescent protein such that a fourth population of insects comprising the male insects of said second population is produced;
(e) crossing said third and fourth population of insects to produce a fifth population of insects comprising male and female insects; and
(f) selecting humanized male insects comprising said human gene of interest from said fifth population of insects.

152. The method of claim 151, wherein said regulatable promoter is selected from the group consisting of heat shock promoter, Gal40, Gal80, Tet, and RU486.

153. The method of claim 151, wherein said insects of said fifth population are insect embryos.

154. The method of claim 151, wherein said male insects of said fifth population express said fluorescent protein.

155. The method of claim 151, wherein step (f) comprises selecting male insects which express said fluorescent protein.

156. The method of claim 155 wherein said step (f) comprises selecting male insects using flow cytometry.

157. The method of claim 156, wherein said flow cytometry is performed using a complex object parametric analyzer and sorter

158. The method of claim 151, wherein said insects are selected from the group consisting of Drosophila, silkworm, and mosquito.

159. The method of claim 151, wherein said pro-apoptotic gene is selected from the group consisting of head involution defective, reaper, grim, hid-ala, ICE, and ced-3.

160. The method of claim 151, wherein said sequence encoding a fluorescent protein encodes a green fluorescent protein.

161. The method of claim 151, wherein said human gene of interest is a neurodegenerative disease gene.

162. A male non-human animal comprising a pro-apoptotic gene operably linked to a regulatable promoter integrated into the Y chromosome, wherein said regulatable promoter is not a heat-shock promoter.

163. A male non-human animal comprising a pro-apoptotic gene operably linked to a regulatable promoter integrated into the Y chromosome, and further comprising integrated into its genome, a nucleic acid sequence encoding Gal4 operably linked to a neuronal or glial-specific promoter.

164. A male insect comprising a pro-apoptotic gene operably linked to a regulatable promoter integrated into the Y chromosome, and further comprises, integrated into the genome of said male insect a nucleic acid sequence encoding Gal4 operably linked to a neuronal or glial-specific promoter.

165. A male Drosophila comprising a pro-apoptotic gene operably linked to a regulatable promoter integrated into the Y chromosome and further comprises, integrated into the genome of said male Drosophila a nucleic acid sequence encoding Gal4 operably linked to a neuronal or glial-specific promoter

166. A female non-human animal comprising an attached-X chromosome, and wherein a pro-apoptotic gene is integrated into said attached-X chromosome.

167. A female non-human animal comprising an attached-X chromosome, wherein a pro-apoptotic gene is integrated into said attached-X chromosome, and wherein said female animal further comprises an upstream activator sequence operably linked to a heterologous gene of interest.

168. A population of female non-human animals comprising an attached-X chromosome, wherein a pro-apoptotic gene is integrated into said attached-X chromosome, and wherein said female animal further comprises an upstream activator sequence operably linked to a heterologous gene of interest, and further comprises a sequence encoding a fluorescent protein integrated into a sex chromosome.

169. A female non-human animal comprising an attached-X chromosome and wherein a pro-apoptotic gene is integrated into said attached-X chromosome, and wherein said female animal further comprises an upstream activator sequence operably linked to a heterologous gene of interest and wherein said female animal further comprises a sequence encoding a fluorescent protein integrated into a sex chromosome and wherein said female animal further comprises, integrated into the X chromosome, a female sterile mutation.

170. A female insect comprising an attached-X chromosome, wherein a pro-apoptotic gene is integrated into said attached-X chromosome.

171. A female Drosophila comprising an attached-X chromosome, and wherein a pro-apoptotic gene is integrated into said attached-X chromosome.
